(19) United States
(12) Patent Application Publication
(10) Pub. No.: US 2024/0272018 A1
(43) Pub. Date: Aug. 15, 2024

(54) MAGNETIC SENSORS AND METHODS OF MAKING AND USING THEREOF

(71) Applicant: UNIVERSITY OF KANSAS, Lawrence, KS (US)

(72) Inventors: Cory BERKLAND, Wildwood, MO (US); Jonathan MILLER, Lawrence, KS (US)

(21) Appl. No.: 18/565,719

(22) PCT Filed: May 31, 2022
(86) PCT No.: PCT/US2022/031608

§ 371 Date: Nov. 30, 2023

Related U.S. Application Data
(60) Provisional application No. 63/195,115, filed on May 31, 2021.

(51) Int. Cl. G01L 1/12; A61B 5/05; H01F 7/08

(52) U.S. Cl. CPC G01L 1/122; A61B 5/05; H01F 7/081; A61B 2562/0223

(57) ABSTRACT

Described herein are magnetic sensors (e.g., force sensors) as well as methods of making and using thereof The magnetic sensors can employ a soft magnetic composite (e.g a composite comprising a population of magnetic particles dispersed within an elastomeric resin) paired with a magnetometer. These sensors can overcome many of the traditional shortcomings that have hampered the effectiveness of existing compression sensors in certain applications, including large size, a lack of 3-dimensional sensing capacity, need for sensors to incorporate rigid components, and/or signal quality issues associated with the orientation or deformation of soft composites under compression.

### CROSS-REFERENCE TO RELATED APPLICATIONS

This application claims benefit of U.S. Provisional Application No. 63/195,115, filed May 31, 2021, which is hereby incorporated herein by reference in its entirety.

### STATEMENT REGARDING FEDERALLY SPONSORED RESEARCH OR DEVELOPMENT

This disclosure was made with Government Support under Grant No. DE-NA-0002893 awarded by Department of Energy. The Government has certain rights to this disclosure.

### BACKGROUND

Soft compression sensors are being pursued in diverse fields, including soft robotics (Rosle et al. 2019), healthcare (Biswas et al. 2020), biomechanics (Low et al. 2015), and others. Sensors composed of silicone rubber or other soft material create a pliable interface, enabling event detection while protecting underlying surfaces. Wearable sensors, for example, may utilize soft materials to render devices imperceivable to the wearer. One type of sensor concept utilizes rigid permanent magnets embedded within silicone or silicone-magnetic powder composites paired with a magnetometer (Wang et al. 2016a; Mirzanejad and Agheli 2019; Hellebrekers et al. 2019; Rosle et al. 2019).

While such sensors may offer an inexpensive, wireless sensor platform, existing designs fail to adequate detect dynamic material compression events. Therefore, improved sensor designs are needed.

### SUMMARY

Described herein are magnetic sensors (e.g., force sensors) as well as methods of making and using thereof. The magnetic sensors can employ a soft magnetic composite (e.g., a composite comprising a population of magnetic particles dispersed within an elastomeric resin) paired with a magnetometer. These sensors can overcome many of the traditional shortcomings that have hampered the effectiveness of existing compression sensors in certain applications, including large size, a lack of 3-dimensional sensing capacity, need for sensors to incorporate rigid components, and/or signal quality issues associated with the orientation or deformation of soft composites under compression. As a result, the sensors described herein can be utilized in wearable technology applications.

Provided herein are force sensors including a magnetic actuator having a proximal end and a distal end; a magnetometer operatively positioned in proximity to the distal end of the magnetic actuator; and a spacer disposed between the magnetometer and the distal end of the magnetic actuator, thereby creating a standoff distance between the magnetometer and the distal end of the magnetic actuator. In some embodiments, the magnetic actuator includes an elastomeric resin; and a population of magnetic particles dispersed within the elastomeric resin. In some embodiments, the spacer can be an elastomeric spacer.

In some embodiments, the force sensor includes a magnetic actuator having a proximal end and a distal end, the magnetic actuator including an elastomeric resin; and a population of magnetic particles dispersed within the elastomeric resin; a magnetometer operatively positioned in proximity to the distal end of the magnetic actuator; and a spacer disposed between the magnetometer and the distal end of the magnetic actuator, thereby creating a standoff distance between the magnetometer and the distal end of the magnetic actuator.

In some embodiments, the force sensor includes a magnetic actuator having a proximal end and a distal end; a magnetometer operatively positioned in proximity to the distal end of the magnetic actuator; and an elastomeric spacer disposed between the magnetometer and the distal end of the magnetic actuator, thereby creating a standoff distance between the magnetometer and the distal end of the magnetic actuator.

In some embodiments, the force sensor can include a magnetic actuator having a proximal end and a distal end; a magnetometer operatively positioned in proximity to the distal end of the magnetic actuator; and an elastomeric housing enclosing at least a portion of the magnetic actuator and extending beyond the distal end of the magnetic actuator, thereby creating a standoff distance between the magnetometer and the distal end of the magnetic actuator. In some embodiments, no elastomeric housing is disposed between the magnetometer and the distal end of the magnetic actuator.

In some embodiments, the magnetic actuator and the magnetometer can be sized relative to one another such that a force applied to the magnetic actuator in a x-y plane relative to the magnetometer, along a z-axis relative to the magnetometer, or any combination thereof produces a magnetic field response that is increasing and proportionate or decreasing and proportionate or decreasing and proportionate to the applied force.

In some embodiments, the magnetic actuator and the magnetometer can be sized relative to one another such that compression of the magnetic actuator under an applied force along the z-axis relative to the magnetometer produces a magnetic field response that is increasing and proportionate or decreasing and proportionate or decreasing and proportionate to the applied force.

In some embodiments, the magnetic actuator and the magnetometer can be sized relative to one another such that shear of the magnetic actuator under an applied force in a x-y plane relative to the magnetometer produces a magnetic field response that is increasing and proportionate or decreasing and proportionate or decreasing and proportionate to the applied force.

In some embodiments, the force sensor can include two or more magnetic actuators, each magnetic actuator having a proximal end and a distal end; a magnetometer operatively positioned in proximity to the distal end of the one or more magnetic actuators; and a spacer disposed between the magnetometer and the distal end of the one or more magnetic actuators, thereby creating a standoff distance between the magnetometer and the distal end of the one or more magnetic actuators.

In some embodiments, the two or more magnetic actuators and the magnetometer are sized relative to one another such that a force applied to the two or more magnetic actuators in a x-y plane relative to the magnetometer, along a z-axis relative to the magnetometer, or any combination thereof produces a magnetic field response that is increasing and proportionate or decreasing and proportionate or decreasing and proportionate to the applied force. In some embodiments, each of the magnetic actuators are adjacent to each other. In some embodiments, the magnetometer is operatively positioned in proximity to the distal end of the magnetic actuators. In some embodiments, the sensor further comprising a rigid spacer disposed between the magnetometer and the distal end of the two or more magnetic actuators, thereby creating a distance between the magnetometer and the distal end of the two or more magnetic actuators, wherein the rigid spacer is formed from a rigid material such as hard plastic, wood, glass, non-magnetic metal, or a material with a Shore A Hardness of greater than 70 and/or a Shore D Hardness of greater than 10.

It will be understood that embodiments employing a rigid spacer can be used, for example, for remote sensing, or sensing through a wall, or across empty air space. An example would be sensing through a protecting casing, where the magnetometer is outside the casing and the elastomeric portion of the sensor is inside the casing. In these examples, the wall of the casing can function as the rigid spacer. As such, the rigid spacer need not be exclusively part of the sensor. In some embodiments, a portion of the spacer can be a void space (e.g., an air space) as well.

In some embodiments, the force sensor can include a magnetic actuator, having a proximal end and a distal end; two or more magnetometers operatively positioned in proximity to the distal end of the magnetic actuator; and a spacer disposed between the two or more magnetometers and the distal end of the magnetic actuator, thereby creating a standoff distance between the one or more magnetometers and the distal end of the magnetic actuator.

In some embodiments, the magnetic actuator and the two or more magnetometers are sized relative to one another such that a force applied to the magnetic actuator in a x-y plane relative to the two or more magnetometers, along a z-axis relative to the two or more magnetometers, or any combination thereof produces a magnetic field response that is increasing and proportionate or decreasing and proportionate to the applied force. In some embodiments, each of the magnetometers are adjacent to each other.

In some embodiments, the magnetic actuator can be a substantially circular horizontal cross-section, cylindrical shape or a substantially conical shape. In some embodiments, the standoff distance can be from greater than 0 mm to 5 mm.

In some embodiments, the spacer can be formed from an elastomeric resin, a rigid material, or any combination thereof. In some embodiments, when the spacer is formed from an elastomeric resin, the spacer includes a portion of a housing that partially or completely encloses the magnetic actuator. In some embodiments, the elastomeric spacer includes a portion of a housing that partially or completely encloses the magnetic actuator. In some embodiments, the elastomeric resin further comprises a non-magnetic filler, such as silica particles. In some embodiments, the sensor can further include a rigid spacer disposed between the magnetometer and the distal end of the elastomeric spacer, thereby creating a standoff distance between the magnetometer and the distal end of the elastomeric spacer, wherein the rigid spacer is formed from a rigid material such as hard plastic, wood, glass, non-magnetic metal, or a material with a Shore A Hardness of greater than 70 and/or a Shore D Hardness of greater than 10. In some other embodiments, the sensor further comprises a rigid spacer disposed between the magnetometer and the distal end of the elastomeric housing, thereby creating a standoff distance between the magnetometer and the distal end of the elastomeric housing, wherein the rigid spacer is formed from a rigid material such as hard plastic, wood, glass, non-magnetic metal, or a material with a shore hardness of greater than 70A or greater than 10D.

In some embodiments, the magnetic particles comprise magnetic microparticles. In some embodiments, the magnetic microparticles have an average particle size of from 1 micron to 150 microns (e.g., 1 micron to 50 microns). In some embodiments, the magnetic particles comprise magnetic nanoparticles. In some embodiments, the magnetic nanoparticles have an average particle size of from 50 nm to less than 1 micron, such as from 50 nm to 500 nm. In some embodiments, the magnetic particles comprise anisotropic magnetic particles.

In some embodiments, the magnetic particles are present in the elastomeric resin in an amount of from 0.1% by weight to 90% by weight, based on the total weight of the elastomeric resin, such as from 50% by weight to 90% by weight, from 40% by weight to 80% by weight, from 30% to 70% by weight, from 20% to 60% by weight, from 15% to 50% by weight, from 0.1% to 50% by weight, from 0.1% to 40% by weight, from 0.1% to 30% by weight, from 0.1% to 20% by weight, from 0.1% by weight to 10% by weight, 0.1% by weight to 5% by weight, from 0.1% by weight to 2.5% by weight, or from 0.1% by weight to 1% by weight, based on the total weight of the elastomeric resin.

In some embodiments, dipoles of the magnetic particles are aligned and/or oriented within the magnetic actuator. In some embodiments, dipoles of the magnetic particles are aligned and/or oriented within the magnetic actuator when the magnetic actuator is compressed by from 10% to 60% under an applied force.

In some embodiments, the sensor further comprises a microcontroller, a processor, or a combination thereof operatively coupled to the magnetometer and configured to calculate a force applied to the magnetometer based on a measurement of a change in magnetic field strength.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows images of A) the 3D-printed forms used to make the silicone sensors, B) the magnetometer used in the study, and C) an example of several magnetic sensors such as cylindrical and conical magnetic elements varied in diameter and height. Depiction of the magnetization of a sensor between two magnets while D) unstrained and E) while strained by the attractive force of the magnets. Pictures of the experimental set ups for F) compression testing, and G) shear testing. All sensor designs have magnetic element positioned away from magnetometer such that a pure rubber gap exists between the magnetic element and the magnetometer.

FIGS. 2A-2D show the Z-axis magnetic field response plotted against time during compression testing of FIG. 2A) a 2×4-cylinder sensor, FIG. 2B) a 2×2-cylinder, FIG. 2C) a 3×3-cylinder, and FIG. 2D) a 5×4-cylinder sensor. Each graph displays data from trials performed with the sensors magnetized while unstrained and from trials performed with the sensors magnetized while strained by the attractive force of the magnets.

FIGS. 3A-3D show the magnetic field response in the X, Y and Z-axes plotted against time during FIG. 3A) shear testing and FIG. 3B) compression testing of a sensor with a 2×4-cylinder sensor, as well as FIG. 3C) shear testing and FIG. 3D) compression testing of a 4×4-cylinder sensor.

FIGS. 4A-4B show the Z-axis magnetic field response plotted against FIG. 4A) displacement and FIG. 4B) force during the increasing compression phase of compression testing for a 2×4-cylinder sensor, as well as a 5×4-cylinder and a 3×3-cone sensor.

FIG. 4C is a plot showing the relationship between the peak magnitude and the volume of the magnetic element of the sensor.

FIG. 4D is a plot showing the relationship between linearity in terms of force and the volume of the magnetic element of the sensor.

FIGS. 5A-5B show the magnetic field response in the Z-axis plotted against time during compression testing of FIG. 5A) 2×4-cylinder sensors comprising 50%, 67% and 80% magnetic filler, as well as FIG. 5B) 2.5×4-cylinder sensors comprising 67% and 80% magnetic filler.

FIGS. 6A-6B show the magnetic field response vs. strain during compression testing for FIG. 6A) a 2×4-cylinder sensor (4 mm total height) with 0-2 mm of silicone spacers between the sensor and the magnetometer during testing, and FIG. 6B) a 3×4-cylinder sensor (4 mm total height) with 0-2.5 mm of silicone spacers between the sensor and the magnetometer during testing.

FIG. 6C shows an image of the set-up for testing the effect of additional remote distance between the sensor and the magnetometer via rigid plastic spacers and an inset graph as an example of the data collected during these tests.

FIG. 6D is a plot of the magnetic field response vs. strain during compression testing for a 3×4-cylinder sensor (5 mm total height) with 0-2 mm of rigid plastic spacers between the sensor and the magnetometer during testing.

FIG. 7 illustrates the deformation of sensors with magnetic elements 2 mm in diameter (A-B) and with 3 mm diameter (C-D) while they are compressed on top of a surface mounted MLX90393 magnetometer such as in the current study. The 2 mm diameter of the magnetic element in the sensor in A is shown to increase to 3.36 mm in B when the sensor is compressed to a 50% strain. The 3 mm diameter of the magnetic element in the sensor in C is shown to increase to 4.53 mm in D when the sensor is compressed to a 50% strain.

FIG. 8 shows images of A) the sensor being integrated into an existing pad of a football helmet, B) of the helmet after integration of the sensor and mounting of the microcontroller module, and C) a helmet-force plate impact trial performed at a slight rightward angle. D) Force (N) plotted against time (s) from the X- and Z-axes of the raw force plate data, the force plate data after downsampling, and the in-helmet sensor data from the helmet-force plate impact pictured in C.

FIGS. 9A-9C show force (N) plotted against time (s) from (FIG. 9A) the Z-axis of the in-helmet sensor and the force plate during helmet-force plate impacts from a predominantly straight forward angle, (FIG. 9B) the X-axis of the in-helmet sensor and the force plate during helmet-force plate impacts from a rightward angle, and (FIG. 9C) a leftward angle.

FIGS. 10A-10B show force (N) and compressive displacement (mm) plotted against time (s) for an example sensor. FIG. 10A shows a 2.5 mm by 4 mm cylinder 80% filler sensor performance calibration equation. FIG. 10B shows 2.5 mm by 4 mm cylinder 80% filler sensor performance force data.

FIG. 11 illustrates a piston design.

FIG. 12 shows magnetic field strength plotted against time for magnetized 5×4 normal or z axis sensing (large volume example) while unstrained or strained at approximately 40%. Magnitude increases from 422 uT to 766 uT (positive direction only). Positive response only for first approximately 1 mm compression. Large magnetic volumes cause signal quality issues.

FIG. 13 shows magnetic field strength plotted against time for magnetized 2×4 normal or z axis sensing while unstrained or strained. Magnitude increases from 1684 uT to 2747 uT.

FIG. 14 shows images of the setup to test the response of a force sensor to different vertical and horizontal remote distances when force was applied in a vertical and/or horizontal orientation.

FIG. 15A shows the magnetic field response plotted against time for a 10×5 mm cylinder cushion with a 2.5×4 cylinder magnetic element (80% Nd) using different vertical remote distances from 2 mm to 10 mm.

FIG. 15B shows the magnetic field response plotted against time for a 10×5 mm cylinder cushion with a 2.5×4 cylinder magnetic element (80% Nd) using vertical remote distance of 10 mm.

FIG. 16 shows magnetic field response plotted against time for a 2.5×4 mm, 80% Nd magnetic element using different horizontal remote distances for gap from rubber cushion to magnetometer board 2-7 mm (true gap from magnetometer to magnetic element 5.75-10.75 mm).

FIG. 17 shows magnetic field response plotted against time for a 5×4 mm cylinder, 80% Nd magnetic element using different horizontal remote distances for gap from rubber cushion to magnetometer board 2 mm to 7 mm (true gap from magnetometer to magnetic element 5.75-10.75 mm). Within both graphs one data plot immediately returns to baseline. This occurred due to a failure of the mechanical testing unit, but demonstrates minimal time lag between the removal of force from the sensor, and the sensor's return to a baseline signal.

FIG. 18 shows magnetic field response plotted against time for a piston design setup with a 10×5 mm cylinder cushion and a 2.5×4 mm cylinder magnetic element (80% Nd) when force was applied on vertical orientations with no gap.

FIG. 19 shows magnetic field response plotted against time for a piston design setup with a 10×5 mm cylinder cushion and a 2.5×4 mm cylinder magnetic element (80% Nd) when force was applied on vertical orientations with different vertical remote distances from 2 mm to 5 mm.

FIG. 20 shows magnetic field response plotted against time for a 3 mm total height rubber cushion and a 2 mm diameter by 2-3 mm height cylinder magnetic element.

FIG. 21 shows images of example 3D printed magnetic sensors.

FIG. 22 shows images of example 3D printed magnetic sensors with different dimensions including 4×4 mm, 3×4 mm, and 2.4×4 mm.

FIG. 23 shows magnetic field response plotted against time for 3D printed magnetic sensors with different dimensions including 4×4 mm, 3×4 mm, and 2.4×4 mm.

FIGS. 24A-24B show a vertical cross section (FIG. 24A) and horizontal cross section (FIG. 24B) of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In this embodiment, the magnetic actuator (101) has a substantially cylindrical shape. In some embodiments, the plate (106) can be absent.

FIG. 25A-25B show a vertical cross section (FIG. 25A) and horizontal cross section (FIG. 25B) of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In this embodiment, the magnetic actuator (101) has a substantially rectangular cuboid shape. In some embodiments, the plate (106) can be absent.

FIG. 26A-26B show a vertical cross section (FIG. 26A) and horizontal cross section (FIG. 26B) of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In this embodiment, the magnetic actuator (101) has a substantially conical shape. In some embodiments, the plate (106) can be absent.

FIG. 27A-27B show a vertical cross section (FIG. 27A) and horizontal cross section (FIG. 27B) of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In this embodiment, the magnetic actuator (101) has a substantially spherical or ovoid shape. In some embodiments, the plate (106) can be absent.

FIG. 28 shows a vertical cross section of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In this embodiment, the largest cross-sectional dimension of the elastomeric housing (111) is larger than the largest cross-sectional dimension of the magnetometer (110) when the magnetic actuator (101) is not subjected to an applied force. In some embodiments, the plate (106) can be absent.

FIG. 29 shows a vertical cross section of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, the elastomeric housing (104) does not completely enclose the magnetic actuator (101). However, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In some embodiments, the plate (106) can be absent.

FIG. 30 shows a vertical cross section of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, the elastomeric housing (104) does not completely enclose the magnetic actuator (101). The elastomeric housing (104) extends beyond the distal end (108) of the magnetic actuator (101), thereby creating a standoff distance (103) between the magnetometer (102) and the distal end (108) of the magnetic actuator (101). In this embodiment, no elastomeric housing is disposed in a region (112) between the magnetometer (102) and the distal end (108) of the magnetic actuator (101). In FIG. 30, the elastomeric housing (104) does not extend beyond and enclose the proximal end (107) of the magnetic actuator (101). However, it some embodiments, the elastomeric housing (104) extends beyond and encloses the proximal end (107) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In some embodiments, the plate (106) can be absent.

FIG. 31 shows a vertical cross section of an example force sensor (100) including a magnetic actuator (101) having a proximal end (107) and a distal end (108), a magnetometer (102) operatively positioned in proximity to the distal end (108) of the magnetic actuator (101), and a plate (106). In this example, an elastomeric spacer (105) is disposed between the magnetometer (102) and the distal end (108) of the magnetic actuator (101). The elastomeric spacer (105) creates a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force.

FIG. 32 shows a vertical cross section of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a first portion (114) of a standoff distance (103) between the magnetometer (102) and the distal end (108) of the magnetic actuator (101). The sensor further includes a rigid spacer (113) disposed between the magnetometer (102) and the distal end (108) of the magnetic actuator (101), such that the rigid spacer (113) creates a second portion (115) of a standoff distance (103) between the magnetometer (102) and the distal end (108) of the magnetic actuator (101). In this example, the largest cross-sectional dimension of the magnetic actuator (109) is smaller than the largest cross-sectional dimension of the magnetometer (110) when the magnetic actuator (101) is not subjected to an applied force. In some embodiments, the plate (106) can be absent.

FIG. 33 shows a vertical cross section of an example force sensor (100) including a magnetic actuator (101) having a proximal end (107) and a distal end (108), an elastomeric housing (104) formed from an elastomeric resin, two magnetometers (102A and 102B) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometers (102A and 102B) and the distal end (108) of the magnetic actuator (101). The elastomeric spacer (105) creates a standoff distance (103) between the magnetometer (102A and 102B) and the distal end (108) of the magnetic actuator (101). In some embodiments, the plate (106) can be absent. In this example, the largest cross-sectional dimension of the magnetic actuator (109) is smaller than the largest cross-sectional distance (117) between the two magnetometers (102A and 102B) when the magnetic actuator (101) is not subjected to an applied force. Using this sensor, rotation can be measured based on the signal detected by the two magnetometers in the x-y axes.

FIG. 34 shows a vertical cross section of an example force sensor (100) including two magnetic actuators (101A and 101B) each having a proximal end (107) and a distal end (108), an elastomeric housing (104) formed from an elastomeric resin, a magnetometer (102) operatively positioned in proximity to the distal end (108) of the two magnetic actuators (101A and 101B), and a plate (106). In this example, a portion of the elastomeric housing (104) extends beyond the distal end (108) of the magnetic actuators, such that a portion of the elastomeric housing forms an elastomeric spacer (105) disposed between the magnetometer (102) and the distal end (108) of two magnetic actuators (101A and 101B). The elastomeric spacer (105) creates a standoff distance (103) between the magnetometer (102) and the distal end (108) of two magnetic actuators (101A and 101B). In some embodiments, the plate (106) can be absent. In this example, the largest cross-sectional dimension of the magnetometer (110) is smaller than the largest cross-sectional distance (116) between the two magnetic actuators (101A and 101B) when the magnetic actuators are not subjected to an applied force. Using this sensor, location of a pinpoint force applied to the top surface of the elastomeric housing can be determined.

FIG. 35 shows a vertical cross section of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the magnetometer (102) and the distal end (108) of the magnetic actuator (101). The elastomeric spacer (105) creates a first portion (114) of a standoff distance (103) between the magnetometer (102) and the distal end (108) of the magnetic actuator (101). The sensor further includes a rigid spacer (113) disposed between the magnetometer (102) and the distal end (108) of the magnetic actuator (101), such that the rigid spacer (113) creates a second portion (115) of a standoff distance (103) between the magnetometer (102) and the distal end (108) of the magnetic actuator (101). The sensor further includes a gap (119) between the magnetometer (102) and the rigid spacer (113), such that the gap (119) creates a third portion (118) of a standoff distance (103) between the magnetometer (102) and the distal end (108) of the magnetic actuator (101). The gap (119) may be a void (e.g., an airspace), or may be filled with any other material, such as a fabric. In this example, the largest cross-sectional dimension of the magnetic actuator (109) is smaller than the largest cross-sectional dimension of the magnetometer (110) when the magnetic actuator (101) is not subjected to an applied force. In some embodiments, the plate (106) can be absent

FIG. 36 shows a vertical cross section of an example force sensor (100) including a magnetic actuator (101) having a proximal end (107) and a distal end (108), an elastomeric housing (104) formed from an elastomeric resin, a magnetometer (102) operatively positioned substantially adjacent to and parallel with the magnetic actuator (101), and a plate (106). In this example, a portion of the elastomeric housing (104) extends beyond the distal end (108) of the magnetic actuator (101), such that a portion of the elastomeric housing forms an elastomeric spacer (105) disposed between the plate (106) and the distal end (108) of the magnetic actuator (101). The elastomeric spacer (105) creates a standoff distance (103) between the plate (106) and the distal end (108) of the magnetic actuator (101).

FIG. 37 shows a vertical cross section of an example force sensor (100) including a magnetic actuator (101) having a proximal end (107) and a distal end (108), an elastomeric housing (104) formed from an elastomeric resin, a magnetometer (102) operatively positioned in proximity to the distal end (108) of the magnetic actuator (101), and a plate (106). In this example, the magnetic actuator (101) abuts the magnetometer (102) (i.e., there is no standoff distance between the magnetometer (102) and the distal end (108) of the magnetic actuator (101)). In this example, the largest cross-sectional dimension of the magnetic actuator (109) is smaller than the largest cross-sectional dimension of the magnetometer (110) when the magnetic actuator (101) is not subjected to an applied force. In some embodiments, the plate (106) can be absent

FIG. 38A shows an image of a flat flexible magnetometer circuit and controller chip.

FIG. 38B shows an image of a sensor formed by combining the silicone sensor component (magnetic actuator enclosed in an elastomeric hosing) with the flat flexible magnetometer circuit.

FIG. 38C is a plot showing the magnetic field response vs. time during several repeated stepwise 1 mm compression cycles performed on a sensor using the flat flexible magnetometer circuit.

FIG. 39 includes images of the qualitative silicone-based adhesive bonding tests (top left and top right), and an image of the three sensors that were constructed using the three different silicone-based adhesives (SILPOXY, MED4-4220, and MED3-4013) to adhere the silicone sensor component (magnetic actuator enclosed in an elastomeric hosing) to the magnetometer circuit board. All three of these adhesives were found to have sufficient bonding strength to join the silicone sensor component to the magnetometer circuit board.

FIG. 40 is a plot sowing magnetic field response vs. time during the 1 mm stepwise compression test for sensor formed by combining the silicone sensor component with the flat flexible magnetometer circuit using various silicone-based adhesives.

FIG. 41 is a three dimensional rendering of an example flat flexible magnetometer circuit and controller chip.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of #10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

By way of non-limiting illustration, examples of certain embodiments of the present disclosure are given below.

### Force Sensor

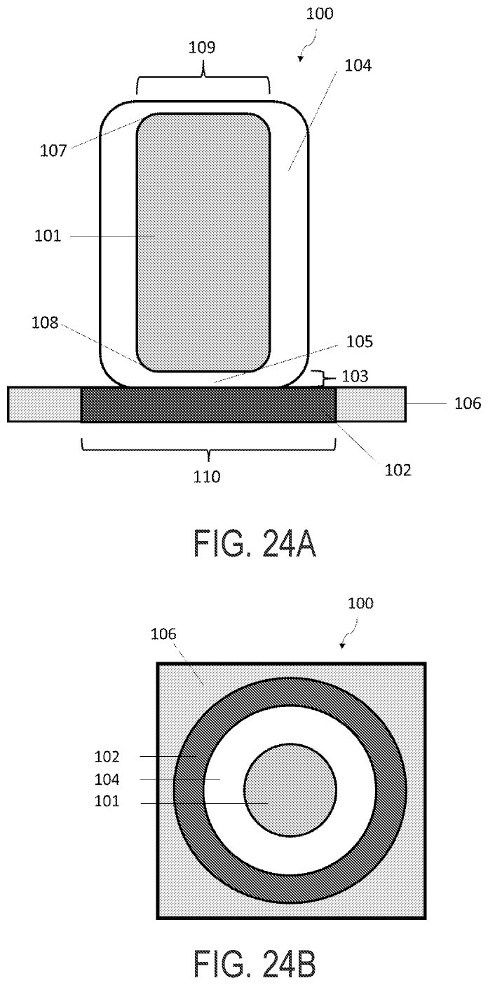

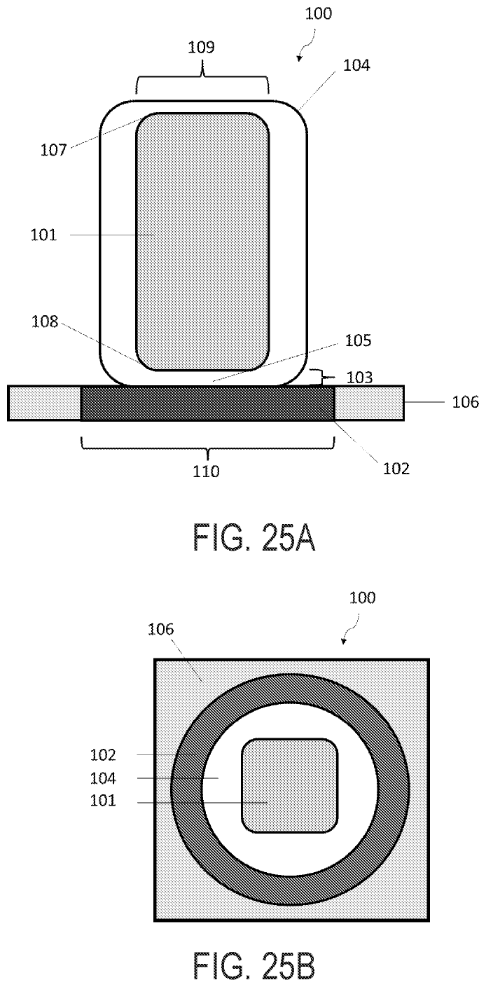

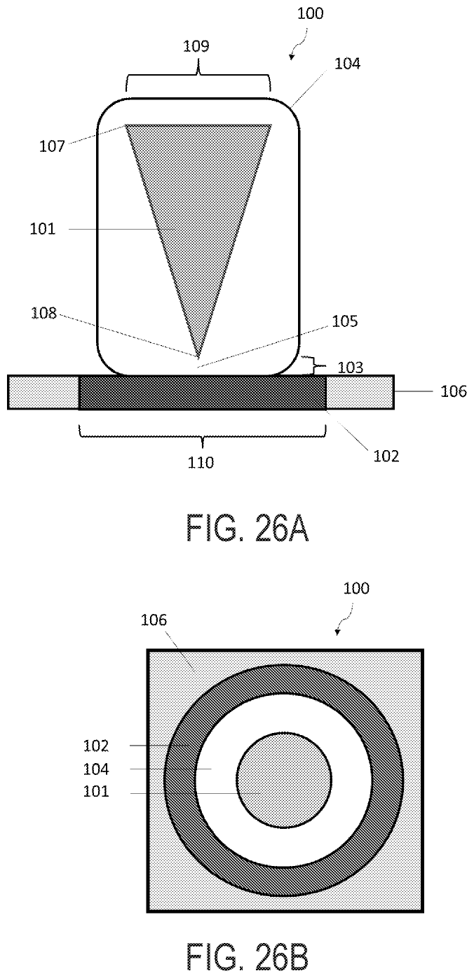

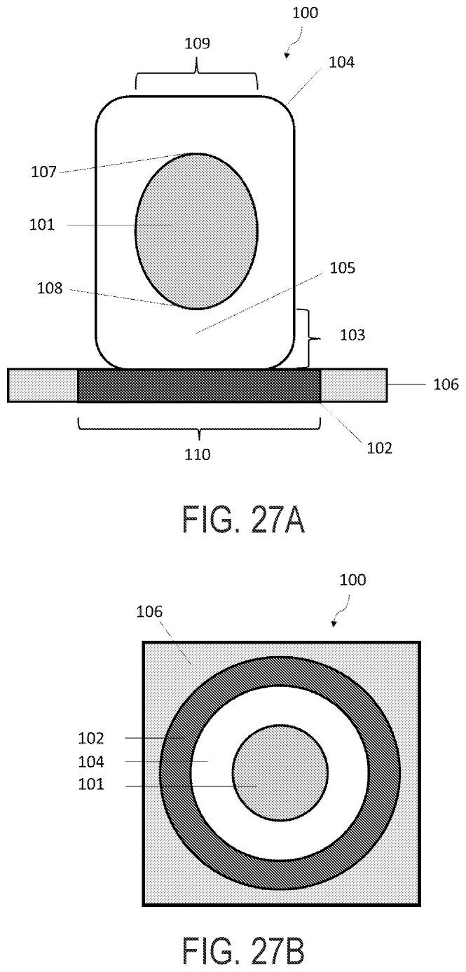

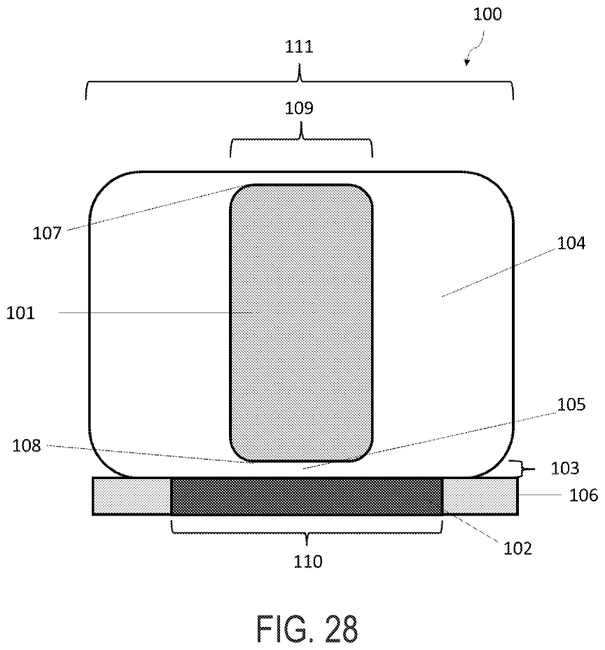

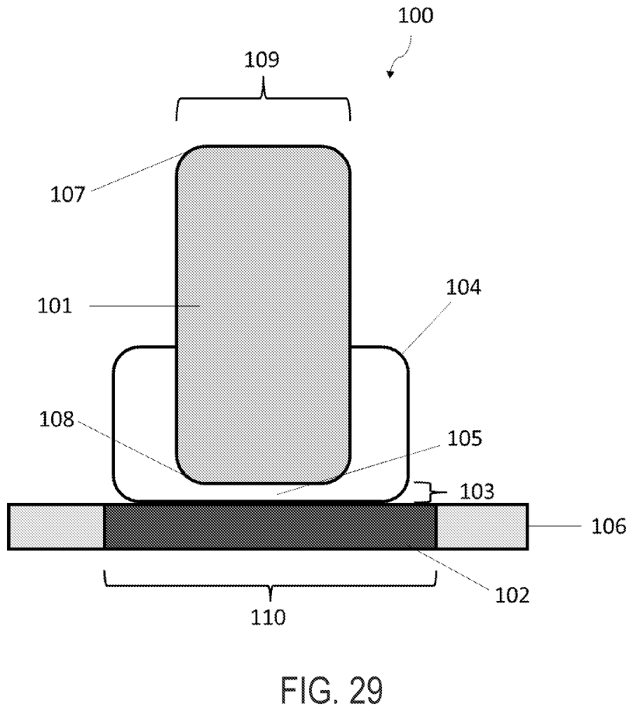

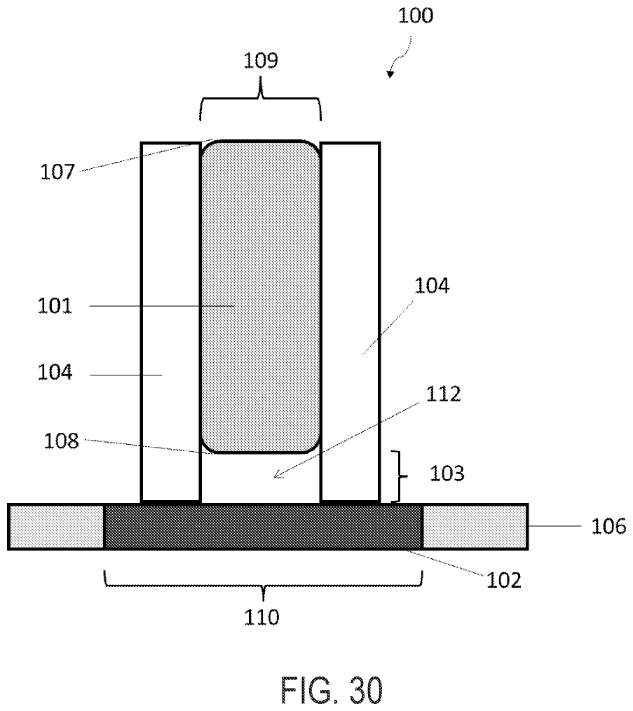

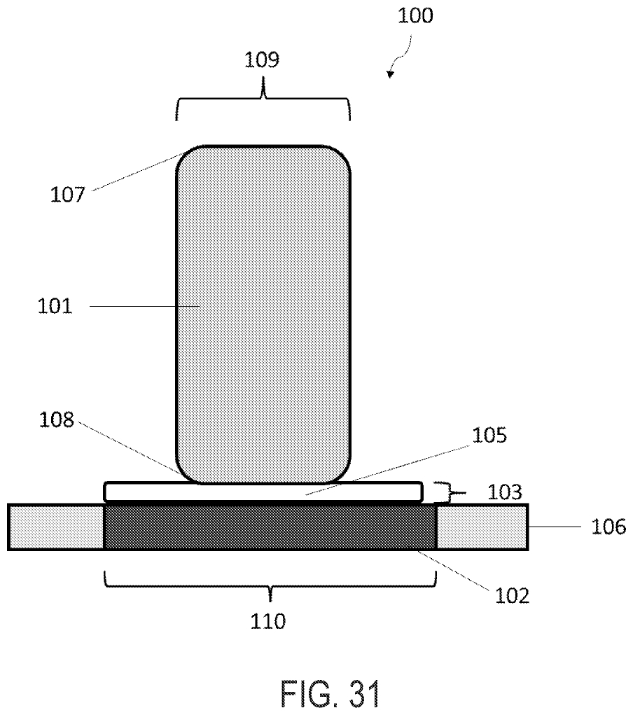

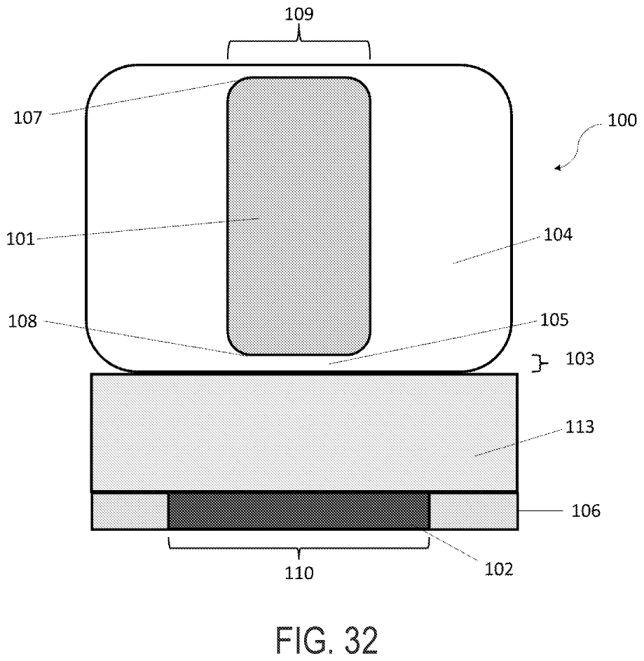

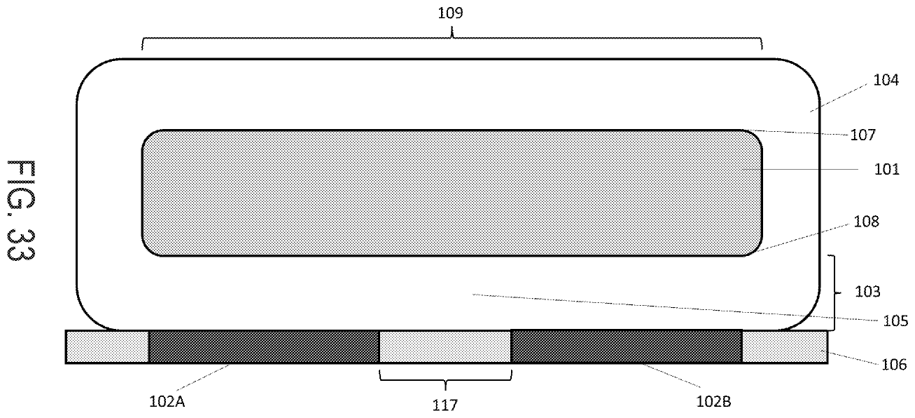

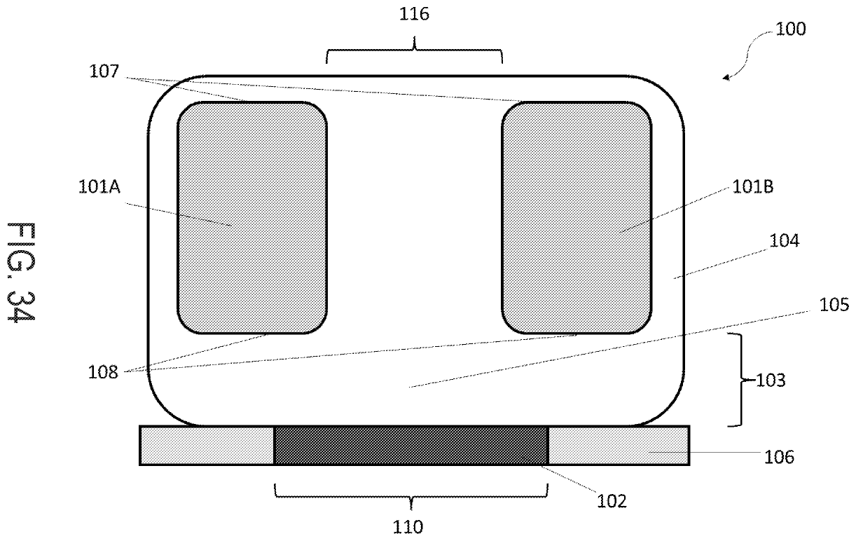

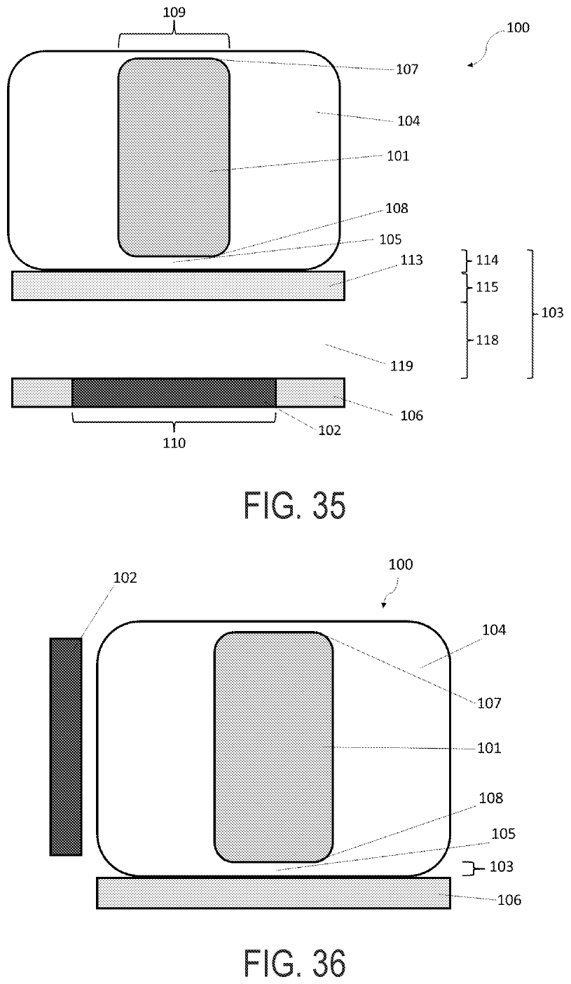

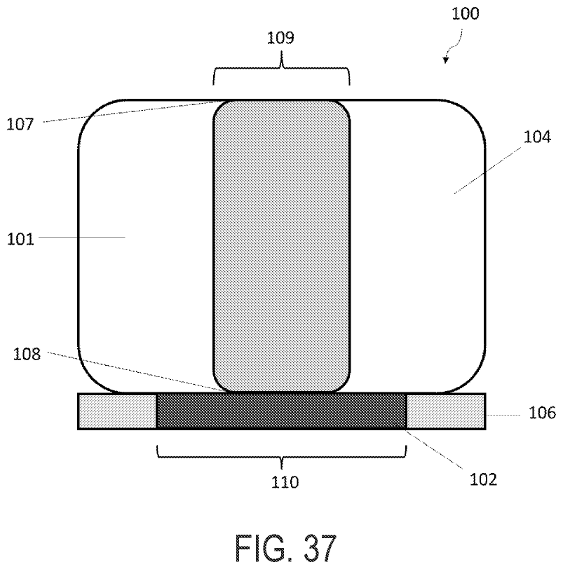

Example force sensors are illustrated in FIGS. 24A-37, which are described in the Description of Drawings above.

Briefly, disclosed herein are force sensors comprising a magnetic actuator having a proximal end and a distal end; a magnetometer operatively positioned in proximity to the distal end of the magnetic actuator; and a spacer disposed between the magnetometer and the distal end of the magnetic actuator, thereby creating a standoff distance between the magnetometer and the distal end of the magnetic actuator. In some embodiments, the magnetic actuator includes an elastomeric resin; and a population of magnetic particles dispersed within the elastomeric resin. In some embodiments, the spacer can be an elastomeric spacer.

In some embodiments, the force sensor includes a magnetic actuator having a proximal end and a distal end, the magnetic actuator including an elastomeric resin; and a population of magnetic particles dispersed within the elastomeric resin; a magnetometer operatively positioned in proximity to the distal end of the magnetic actuator; and a spacer disposed between the magnetometer and the distal end of the magnetic actuator, thereby creating a standoff distance between the magnetometer and the distal end of the magnetic actuator.

In some embodiments, the force sensor includes a magnetic actuator having a proximal end and a distal end; a magnetometer operatively positioned in proximity to the distal end of the magnetic actuator; and an elastomeric spacer disposed between the magnetometer and the distal end of the magnetic actuator, thereby creating a standoff distance between the magnetometer and the distal end of the magnetic actuator.

In some embodiments, the force sensor can include a magnetic actuator having a proximal end and a distal end; a magnetometer operatively positioned in proximity to the distal end of the magnetic actuator; and an elastomeric housing enclosing at least a portion of the magnetic actuator and extending beyond the distal end of the magnetic actuator, thereby creating a standoff distance between the magnetometer and the distal end of the magnetic actuator. In some embodiments, no elastomeric housing is disposed between the magnetometer and the distal end of the magnetic actuator.

In some embodiments, the magnetic actuator and the magnetometer can be sized relative to one another such that a force applied to the magnetic actuator in a x-y plane relative to the magnetometer, along a z-axis relative to the magnetometer, or any combination thereof produces a magnetic field response that is increasing and proportionate or decreasing and proportionate to the applied force.

In some embodiments, the magnetic actuator and the magnetometer can be sized relative to one another such that compression of the magnetic actuator under an applied force along the z-axis relative to the magnetometer produces a magnetic field response that is increasing and proportionate or decreasing and proportionate to the applied force.

In some embodiments, the force sensor can include two or more magnetic actuators, each magnetic actuator having a proximal end and a distal end; a magnetometer operatively positioned in proximity to the distal end of the one or more magnetic actuators; and a spacer disposed between the magnetometer and the distal end of the one or more magnetic actuators, thereby creating a standoff distance between the magnetometer and the distal end of the one or more magnetic actuators.

In some embodiments, the two or more magnetic actuators and the magnetometer are sized relative to one another such that a force applied to the two or more magnetic actuators in a x-y plane relative to the magnetometer, along a z-axis relative to the magnetometer, or any combination thereof produces a magnetic field response that is increasing and proportionate or decreasing and proportionate to the applied force. In some embodiments, each of the magnetic actuators are adjacent to each other. In some embodiments, the magnetometer is operatively positioned in proximity to the distal end of the magnetic actuators. In some embodiments, the sensor further comprising a rigid spacer disposed between the magnetometer and the distal end of the two or more magnetic actuators, thereby creating a distance between the magnetometer and the distal end of the two or more magnetic actuators, wherein the rigid spacer is formed from a rigid material such as hard plastic, wood, glass, non-magnetic metal, or a material with a Shore A Hardness of greater than 70 and/or a Shore D Hardness of greater than 10.

In some embodiments, the force sensor can include a magnetic actuator, having a proximal end and a distal end; two or more magnetometers operatively positioned in proximity to the distal end of the magnetic actuator; and a spacer disposed between the two or more magnetometers and the distal end of the magnetic actuator, thereby creating a standoff distance between the one or more magnetometers and the distal end of the magnetic actuator.

In some embodiments, the magnetic actuator and the two or more magnetometers are sized relative to one another such that a force applied to the magnetic actuator in a x-y plane relative to the two or more magnetometers, along a z-axis relative to the two or more magnetometers, or any combination thereof produces a magnetic field response that is increasing and proportionate or decreasing and proportionate to the applied force. In some embodiments, each of the magnetometers are adjacent to each other.

In some embodiments, the magnetic field response can increase 5-20% less than the increase proportionate to the applied force.

In some embodiments, the magnetic actuator can have a largest cross-sectional dimension that is smaller than a largest cross-sectional dimension of the magnetometer when the magnetic actuator is not subjected to an applied force. The largest cross-sectional dimension of the magnetic actuator can be from 5% to 80% of the largest cross-sectional dimension of the magnetometer when the magnetic actuator is not subjected to an applied force. In some embodiments, the magnetic actuator can have a largest cross-sectional dimension that is smaller than a largest cross-sectional dimension of the magnetometer when the magnetic actuator is compressed by 40% under an applied force. In some embodiments, the largest cross-sectional dimension of the magnetic actuator can be from 50% to 90% of the largest cross-sectional dimension of the magnetometer when the magnetic actuator is compressed by 40% under an applied force.

In some embodiments, the magnetic actuator can have a largest cross-sectional area that is smaller than a largest cross-sectional area of the magnetometer when the magnetic actuator is not subjected to an applied force. The largest cross-sectional area of the magnetic actuator can be from 50% to 90% of the largest cross-sectional are of the magnetometer when the magnetic actuator is not subjected to an applied force. In some embodiments, the magnetic actuator has a largest cross-sectional area that is smaller than a largest cross-sectional area of the magnetometer when the magnetic actuator is compressed by 40% under an applied force. In some embodiments, the largest cross-sectional area of the magnetic actuator is from 50% to 90% of the largest cross-sectional area of the magnetometer when the magnetic actuator is compressed by 40% under an applied force.

In some embodiments, the magnetic actuator and the magnetometer are sized relative to one another such that compression of the magnetic actuator under a window of applied forces ranging from an applied force effective to compress the magnetic actuator by 5% to an applied force effective to compress the magnetic actuator by 40% produces a magnetic field response that is increasing and proportionate or decreasing and proportionate to the applied force across the window of applied forces. In some embodiments, the magnetic actuator and the magnetometer are sized relative to one another such that compression of the magnetic actuator under a window of applied forces ranging from an applied force effective to compress the magnetic actuator by 3% to an applied force effective to compress the magnetic actuator by 20% produces a magnetic field response that is increasing and proportionate or decreasing and proportionate to the applied force across the window of applied forces.

In some embodiments, a force applied to the magnetic actuator in the x-y plane relative to the magnetometer produces a magnetic field response that is increasing and linear relative to the applied force. In some embodiments, a force applied to the magnetic actuator along the z-axis relative to the magnetometer produces a magnetic field response that is increasing and linear relative to the applied force. In some embodiments, a force applied to the magnetic actuator in the x-y plane relative to the magnetometer produces a magnetic field response that is increasing and linear relative to the applied force and a force applied to the magnetic actuator along the z-axis relative to the magnetometer produces a magnetic field response that is increasing and linear relative to the applied force.

In some embodiments, the magnetic actuator and the magnetometer can be sized relative to one another such that shear of the magnetic actuator under an applied force in a x-y plane relative to the magnetometer produces a magnetic field response that is increasing and proportionate or decreasing and proportionate to the applied force. The magnetic actuator can have a largest cross-sectional dimension that is smaller than a largest cross-sectional dimension of the magnetometer when the magnetic actuator is not subjected to an applied force. In some embodiments, the largest cross-sectional dimension of the magnetic actuator can be from 5% to 80% of the largest cross-sectional dimension of the magnetometer when the magnetic actuator is not subjected to an applied force. In some embodiments, the magnetic actuator can have a largest cross-sectional dimension that is smaller than a largest cross-sectional dimension of the magnetometer when the magnetic actuator is sheared by 40% under an applied force. In some embodiments, the largest cross-sectional dimension of the magnetic actuator can be from 50% to 90% of the largest cross-sectional dimension of the magnetometer when the magnetic actuator is sheared by 40% under an applied force.

In some embodiments, the magnetic actuator can have a largest cross-sectional area that is smaller than a largest cross-sectional area of the magnetometer when the magnetic actuator is not subjected to an applied force. The largest cross-sectional area of the magnetic actuator can be from 50% to 90% of the largest cross-sectional are of the magnetometer when the magnetic actuator is not subjected to an applied force. In some embodiments, the magnetic actuator has a largest cross-sectional area that is smaller than a largest cross-sectional area of the magnetometer when the magnetic actuator is sheared by 40% under an applied force. In some embodiments, the largest cross-sectional area of the magnetic actuator is from 50% to 90% of the largest cross-sectional area of the magnetometer when the magnetic actuator is sheared by 40% under an applied force.

In some embodiments, the magnetic actuator and the magnetometer are sized relative to one another such that shear of the magnetic actuator under a window of applied forces ranging from an applied force effective to induce a shear strain of the magnetic actuator by 5% to an applied force effective to induce a shear strain of the magnetic actuator by 40% produces a magnetic field response that is increasing and proportionate or decreasing and proportionate to the applied force across the window of applied forces. In some embodiments, the magnetic actuator and the magnetometer are sized relative to one another such that shear of the magnetic actuator under a window of applied forces ranging from an applied force effective to induce a shear strain of the magnetic actuator by 3% to an applied force effective to induce a shear strain of the magnetic actuator by 20% produces a magnetic field response that is increasing and proportionate or decreasing and proportionate to the applied force across the window of applied forces.

In some embodiments, the magnetic actuator can have a largest cross-sectional dimension of from 1 mm to 25 mm. For example, from 1 mm to 5 mm, from 1 mm to 10 mm, from 1 mm to 15 mm, from 1 mm to 20 mm, from 5 mm to 20 mm, from 5 mm to 15 mm, from 10 mm to 25 mm, or from 10 mm to 20 mm.

In some embodiments, the magnetic actuator can be a substantially circular horizontal cross-section, cylindrical shape or a substantially conical shape. In some embodiments, the magnetic actuator has a substantially circular horizontal cross-section. In some embodiments, the magnetic actuator has a substantially cylindrical shape. In some embodiments, the magnetic actuator has a substantially conical shape.

In some embodiments, the standoff distance can be from greater than 0 mm to 5 mm, such as from greater than 0 mm to 1.5 mm, greater than 0 mm to 3 mm, greater than 1 mm to 5 mm, greater than 1 to 3 mm, or greater than 2 mm to 5 mm. In some embodiments, the standoff distance can be selected to provide a measurable signal such as magnetic field response greater than 100 μT with an applied force. In some embodiments there may be no standoff distance.

In some embodiments, the spacer can be formed from an elastomeric resin, a rigid material, or any combination thereof. In some embodiments, the spacer can be formed from an elastomeric resin. In some embodiments, the spacer can be an elastomeric spacer.

In some embodiments, the elastomeric housing is formed from an elastomeric resin. In some embodiments, the elastomeric spacer can be formed from an elastomeric resin.

In some embodiments, the spacer can be formed from a rigid material. In some embodiments, when the spacer is formed from an elastomeric resin, the spacer includes a portion of a housing that partially or completely encloses the magnetic actuator. In some embodiments, the elastomeric spacer includes a portion of a housing that partially or completely encloses the magnetic actuator.

In some embodiments, the elastomeric resin further comprises a non-magnetic filler, such as silica particles. In some embodiments, the elastomeric resin comprises a crosslinkable composition, such as a crosslinkable silicone composition. In some embodiments, the elastomeric resin comprises (A) a first organosilicon compound having at least two ethylenically unsaturated moieties per molecule; and optionally (B) one or more additional organosilicon compounds. In some embodiments, the sensor can further include a rigid spacer disposed between the magnetometer and the distal end of the elastomeric spacer, thereby creating a standoff distance between the magnetometer and the distal end of the elastomeric spacer, wherein the rigid spacer is formed from a rigid material such as hard plastic, wood, glass, non-magnetic metal, or a material with a Shore A Hardness of greater than 70 and/or a Shore D Hardness of greater than 10. In some other embodiments, the sensor further comprises a rigid spacer disposed between the magnetometer and the distal end of the elastomeric housing, thereby creating a standoff distance between the magnetometer and the distal end of the elastomeric housing, wherein the rigid spacer is formed from a rigid material such as hard plastic, wood, glass, non-magnetic metal, or a material with a shore hardness of greater than 70A or greater than 10D.

In some embodiments, the sensor further comprises a microcontroller, a processor, or a combination thereof operatively coupled to the magnetometer and configured to calculate a force applied to the magnetometer based on a measurement of a change in magnetic field strength.

### Composites Forming the Magnetic Actuator

As discussed above, the magnetic actuator can be formed of a composite that comprises an elastomeric resin; and a population of magnetic particles (e.g., anisotropic magnetic particles) dispersed within the elastomeric resin.

### Magnetic Particles

The magnetic particles can be any suitable magnetic particles.

In some embodiments, the magnetic particles comprise magnetic microparticles. The microparticles can be of any shape, and have one or more dimensions ranging from 1 micron to 150 microns (e.g., from 1 micron to 100 microns, or from 1 micron to 50 microns). In some embodiments, all dimensions can range from 1 micron to 150 microns (e.g., from 1 micron to 100 microns, or from 1 micron to 50 microns).

In some embodiments, the magnetic particles can comprise nanoparticles. The term “nanoparticle,” as used herein, generally refers to a particle of any shape having one or more dimensions ranging from 1 nm up to, but not including, 1 micron.

In some embodiments, the population of magnetic particles are a monodisperse population of magnetic particles. In other embodiments, the population of magnetic particles are a polydisperse population of anisotropic magnetic particles. In some instances where the population of magnetic particles is polydisperse, greater that 50% of the particle size distribution, more preferably 60% of the particle size distribution, most preferably 75% of the particle size distribution lies within 10% of the median particle size.

The magnetic particles can comprise any suitable magnetic material, such as ferromagnetic alloys comprising Fe, Nd, Co, Ni, or combinations thereof. In certain embodiments, the magnetic particles can comprise Ni particles. In some embodiments, the magnetic particles can comprise spherical (or substantially spherical) magnetic particles. In some embodiments, the magnetic particles can comprise cubic magnetic particles. In other embodiments, the magnetic particles can comprise anisotropic magnetic particles. Such particles can be formed using methods known in the art, including synthesis driven by appropriate shaping ligands, template-assisted synthesis, template-assisted electrodeposition, and magnetically directed assembly. Examples of such materials are described, for example, in Lisjak et al. “Anisotropic Magnetic Nanoparticles: A Review of their Properties, Synthesis, and Potential Applications,” Progress in Materials Science, 2018, 95; 286-328 (which is hereby incorporated by reference in its entirety for its description of anisotropic magnetic particles, and which is attached to this filing).

The magnetic particles can be essentially homogeneous throughout, meaning that the composition does not vary throughout the particle cross-section (from the particle surface to the particle center). Alternatively, the magnetic particles can possess a non-homogeneous structure. For example, the particles may possess a core-shell structure, or a multilayer structure (e.g., a magnetic core coated by a non-magnetic shell material).

The magnetic particles may have any desired shape. In certain embodiments, the particles can have a non-spherical shape. As generally used herein, “non-spherical” is used to describe particles having at least one dimension differing from another dimension by a ratio of at least 1:1.10. In one embodiment, the non-spherical particles have at least one dimension which differs from another dimension by a ratio of at least 1:1.25. A wide variety of shapes are considered “non-spherical” shapes. For example, non-spherical particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those illustrated in the figures are also within the scope of the definition for “non-spherical” shapes.

In some embodiments, the magnetic particles can comprise rod-shaped particles. “Rod-shaped,” as used herein, refers to a particle which has an elongated spherical or cylindrical shape (e.g., the shape of a pill) or a flattened rod-shape, such as the shape of a green bean. Rod-shaped particles have an aspect ratio of at least 1.25 (e.g., at least 1.5, at least 2, at least 2.5, or at least 5). “Aspect ratio,” as used herein, refers to the length divided by the diameter of a particle.

In certain embodiments, the particles can be rod-shaped. In some embodiments, the rod-shaped particles can have an aspect ratio, defined as the length of the rod-shaped particle divided by the diameter of the rod-shaped particle, of at least 1.25 (e.g., at least 2.5, at least 5, at least 10, at least 15, at least 25, at least 50, at least 100, at least 150, at least 200, at least 250, or more). In some embodiments, the rod-shaped particles can have an aspect ratio, defined as the length of the rod-shaped particle divided by the diameter of the rod-shaped particle, of 500 or less (e g., 250 or less, 200 or less, 150 or less, 100 or less, 50 or less, 25 or less, 15 or less, 10 or less, 5 or less, or 2.5 or less).

The rod-shaped particles can have an aspect ratio ranging from any of the minimum values described above to any of the maximum values described above. In certain embodiments, the rod-shaped particles can have an aspect ratio of from 1.25 to 500 (e.g., from 5 to 500, from 5 to 250, from 5 to 100, from 5 to 500, from S to 250, or from 5 to 100).

In some embodiments, the rod-shaped particles can have an average diameter of at least 5 nm (e.g., at least 25 nm, at least 50 nm, at least 100 nm, at least 200 nm, at least 300 nm, at least 400 nm, at least 500, at least 600 nm, at least 700 nm, at least 800 nm, or at least 900 nm). In some embodiments, the rod-shaped particles can have an average diameter of 950 nm or less (e.g., 900 nm or less, 800 nm or less, 700 nm or less, 600 nm or less, 500 nm or less, 400 nm or less, 300 nm or less, 200 nm or less, 100 nm or less, 50 nm or less, or 25 nm or less.

The rod-shaped particles can have an average diameter ranging from any of the minimum values described above to any of the maximum values described above. In certain embodiments, the rod-shaped particles can have an average diameter of from 50 nm to 800 nm (e.g., from 50 nm to 500 nm, or from 100 nm to 300 nm).

In some embodiments, the rod-shaped particles can have an average length of at least 500 nm (e g., at least 1 micron, at least 5 microns, at least 10 microns, at least 15 microns, at least 20 microns, at least 25 microns, at least 50 microns, at least 75 microns, at least 100 microns, at least 150 microns, or at least 200 microns). In some embodiments, the rod-shaped particles can have an average length of 250 microns or less (e.g., 200 microns or less, 150 microns or less, 100 microns or less, 75 microns or less, 50 microns or less, 25 microns or less, 20 microns or less, 15 microns or less, 10 microns or less, 5 microns or less, or 1 micron or less).

The rod-shaped particles can have an average length ranging from any of the minimum values described above to any of the maximum values described above. In certain embodiments, the rod-shaped particles can have an average length of from 500 nm to 100 microns (e.g., from 1 micron to 25 microns).

In some embodiments, the magnetic particles can comprise anisotropic magnetic particles. In some embodiments, dipoles of the magnetic particles are aligned and/or oriented within the magnetic actuator. In some embodiments, dipoles of the magnetic particles are aligned and/or oriented within the magnetic actuator when the magnetic actuator is compressed by from 10% to 60% under an applied force.

The magnetic particles can be present in the composition in an amount of from 0.1% by weight to 90% by weight, based on the total weight of the elastomeric resin, such as from 50% by weight to 90% by weight, from 40% by weight to 80% by weight, from 30% to 70% by weight, from 20% to 60% by weight, from 15% to 50% by weight, from 0.1% to 50% by weight, from 0.1% to 40% by weight, from 0.1% to 30% by weight, from 0.1% to 20% by weight, from 0.1% by weight to 10% by weight, 0.1% by weight to 5% by weight, from 0.1% by weight to 2.5% by weight, or from 0.1% by weight to 1% by weight, based on the total weight of the elastomeric resin.

The magnetic particles can be present in the composition in an amount of from 0.01% by volume to 20% by volume (e.g., from 0.01% by volume to 15% by volume, from 0.01% by volume to 10% by volume, from 0.01% by volume to 7.5% by volume, from 0.01% by volume to 5% by volume, from 0.01% by volume to 2.5% by volume, or from 0.01% by volume to 1% by volume), based on the total volume of the composition.

In some embodiments, the magnetic particles can be uniformly dispersed throughout the elastomeric resin. In other embodiments, the magnetic particles can by non-homogenously dispersed throughout the elastomeric resin. For example, the magnetic particles can be at varying concentrations throughout the elastomeric resin (e.g., at a higher concentration at a region in proximity to a magnetometer and at a lower concentration at a region further away from a magnetometer). In some embodiments, a gradient of magnetic particles can be dispersed within the elastomeric resin.

### Elastomeric Resins

The elastomeric resin can comprise an elastomeric resin suitable for use in an additive manufacturing process. Such materials are well known in the art. In some examples, the elastomeric resin can comprise a thermoplastic polymer such as acrylonitrile butadiene styrene (ABS), polyphenylene sulfide (PPS), polyphenylsulfone (PPSU), polyetheretherketone (PEEK), polyurethane (PU), polyetherimide (PEI), polyphenylene ether (PPE), polycarbonate (PC), and combinations thereof. In some embodiments, the elastomeric resin can comprise a crosslinkable composition (e.g., a blend of monomers, oligomers, and/or polymers which can be crosslinked during the additive manufacturing process). Depending on the additive manufacturing process employed, the crosslinkable composition can be selected such that crosslinking can be induced thermally and/or by impinging electromagnetic radiation (e.g., UV and/or visible light). In certain embodiments, the elastomeric resin can comprise a crosslinkable silicone composition. For example, the elastomeric resin can comprise (A) a first organosilicon compound having at least two ethylenically unsaturated moieties per molecule; and optionally (B) one or more additional organosilicon compounds. Suitable silicone compositions are known in the art. See, for example, U.S. Pat. No. 10,155,884 to Dow Silicones Corp., U.S. Patent Application Publication No. 2017/0312981 to Wacker Chemie AG, U.S. Patent Application Publication No. 2018/0370141 to Wacker Chemie AG, U.S. Patent Application Publication No. 2018/0066115 to Wacker Chemie AG, U.S. Patent Application Publication No. 2018/0186076 to Dow Corning Corp., and U.S. Patent Application Publication No. 2019/0100626 to Lawrence Livermore National Security LLC, each of which is hereby incorporated by reference in its entirety. Other suitable elastomeric resins are described, for example, in U.S. Patent Application Publication No. 20160319150 to Cornell University.

Optionally, the composition may further optionally a non-magnetic filler. The non-magnetic filler may be, for example, an organic filler, an inorganic filler, a ceramic powder, or combinations thereof. The organic filler may be a polymer, such as, but not limited to, polystyrene, polyethylene, polypropylene, polysulfone, polyamide, polyimide, polyetheretherketone, etc. The organic filler can also be a smaller molecule either amorphous or crystalline in nature, and can be of in various shapes and sizes. The inorganic filler or ceramic powder can be any inorganic compounds that are compatible with the curing chemistry. Examples include, but are not limited to, silicon dioxide, titanium dioxide, zirconium dioxide, barium titanate, strontium titanate, etc. A mixture of more than one inorganic or organic with inorganic fillers are also suitable.

In embodiments including the non-magnetic filler, the non-magnetic filler can be present as any suitable wt. % of the composition, such as about 0.01 wt. % to about 90 wt. %, about 1 wt. % to about 80 wt. %, about 5 wt. % to about 80 wt. %, about 10 wt. % to about 80 wt. %, about 15 wt. % to about 80 wt. %, about 25 wt. % to about 80 wt. %, about 30 wt. % to about 80 wt. %, about 40 wt. % to about 80 wt. %, about 50 wt. % to about 75 wt. %, about 55 wt. % to about 75 wt. %, about 60 wt. % to about 70 wt. %, alternatively about 0.1 wt. %, 1, 2, 3, 4, 5, 6, 7, 8, 9, 10, 11, 12, 13, 14, 15, 16, 18, 20, 25, 30, 35, 40, 45, 50, 55, 60, 65, or about 70 wt. % or more.

The non-magnetic filler can have any suitable particle size, e.g., the longest dimension of the particle, such as the average longest dimension. For example, the non-magnetic filler can have a primary particle size of about 5 to about 100, about 10 to about 90, about 20 to about 80, about 30 to about 70, about 40 to about 60, or about 50, microns, alternatively 5 microns or less, alternatively 100 microns or more. As used herein, “primary” particle size refers to the actual particles in their un-conglomerated state, which can optionally conglomerate to form larger “secondary” particles.

Any of the compositions may optionally and independently further comprise additional ingredients or components (“additives”). Examples of additional ingredients include, but are not limited to, adhesion promoters; dyes; pigments; anti-oxidants; initiators for crosslinking, carrier vehicles; heat stabilizers; flame retardants; thixotropic agents; flow control additives; inhibitors; extending and reinforcing fillers; and cross-linking agents. One or more of the additives can be present as any suitable wt. % of the composition, such as about 0.1 wt. % to about 15 wt. %, about 0.5 wt. % to about 5 wt. %, or about 0.1 wt. % or less, about 1 wt %, 2, 3, 4, 5, 6, 7, 8, 9, 10, 11, 12, 13, 14, or about 15 wt. % or more of the composition.

### EXAMPLES

### Example 1: Proof of Principle Sensors

### Overview

Detecting the magnitude and rate of compression or shear events can provide valuable data to assess forces experienced by sensitive equipment or even the human body. Compression sensors have historically had limited use in fields such as wearable technology due to the large size of the sensors, lack of 3-dimensional sensing capacity, need for rigid components, or signal quality issues associated with the orientation or deformation of soft composites under compression. In particular, soft magnetic composites paired with a magnetometer to sense material deformation have been hindered by such issues. Here, experiments evaluated soft silicone-magnetic powder composites of various shape, size, magnetic filler concentration, and orientation to a magnetometer to optimize sensor performance. Sensors with smaller diameter (≤2.5 mm) cylinder-shaped magnetic elements exhibited a linear response to compression when paired with a 3 mm×3 mm magnetometer. The soft magnetic elements composed of 80% magnetic powder by weight, the highest concentration tested, had the greatest sensitivity. Parameters from these studies were used to design a sensor integrated into a football helmet, which successfully recorded 3-dimensional force data from head impacts while being worn, demonstrating the potential for such sensors in wearable technology applications.

### Introduction

Soft magnetic sensors operate by detecting magnetic field changes as embedded magnetic material moves in relation to a magnetometer during compression of a soft material. Previous designs for soft magnetic sensors had limitations, which inhibit their use for accurately and reliably detecting forces on sensitive components or in wearable technology applications. For example, most previous sensors utilized rigid permanent magnets (Wang et al. 2016b; Rosle et al. 2019) which create durability issues (Wang et al. 2016a). Large, solid magnets are also not appropriate for applications where forceful contact between the sensor and a person would cause discomfort, such as when embedded within a shoe insole, unless the cushion is made significantly thicker than typical shoe insoles (Johnson and Ozkan; Lam et al. 2017).

Materials that comply when forces are applied and yet maintain a high magnetic flux are desired. Unfortunately, soft sensors composed of silicone-magnetic powder composites have also suffered from signal quality issues (Mirzanejad and Agheli 2019), have lacked the sensitivity necessary for accurate sensing of high forces (Hellebrekers et al. 2019), and none have displayed the capacity for accurately sensing shear forces. Previous reports employed sensors which required operation below certain levels of force before further compression caused the signal to diminish (Mirzanejad and Agheli 2019). A misalignment of magnetic dipoles was hypothesized, since the magnetic element in the sensor was deformed under compression. We hypothesized the geometry of the magnetic elements may also have been too large, since the smallest magnetic elements in the study were capable of sensing the highest forces.

Soft magnetic sensors also present an opportunity to detect shear forces in addition to compression. For example, shear could be detected using triple axis magnetometers paired with an appropriately designed soft magnetic element, although such a sensor has yet to be reported. This is likely due to issues caused by the geometry of the magnetic element or improper orientation of the sensor and magnetic element. Certainly, a soft 3-dimensional force sensor appropriate for wearable technology applications has not been realized.

Therefore, soft magnetic sensors were fabricated with considerations to achieve properties suitable for wearable technology while overcoming signal quality issues present in previous reports. Multiple geometries and magnetic filler concentrations of the magnetic element were investigated. Methods for aligning the dipoles of the magnetic particles embedded in the magnetic element were also explored. The relative position of magnetic elements with respect to the location of a magnetometer was assessed. Finally, design considerations for sensors were compiled and used in a wearable technology application.

### Methods

### Sensor Design and Fabrication

All sensors were 10 mm diameter by 5 mm height cylinders composed of polydimethylsiloxane (PDMS) silicone rubber elastomer (Sylgard 184, 20:1 base to curing agent ratio, Dow Chemical Company; Pevely, MO, USA), with a discreet cylindrical or conical portion of the construct composed of a silicone-magnetic powder composite. The magnetic powder used in the composite was a neodymium iron boron alloy (NdFeB) with an average particle size <10 μm (American Elements, Los Angeles, CA, USA). The geometry of this magnetic element was variable, as the most efficacious geometry for the magnetic element of the sensor was a primary question of the investigation. Sensors were fabricated with cylindrical magnetic elements with diameters of 2, 3, 4, and 5 mm, and heights of 2, 3, and 4 mm, and with conical magnetic elements with base diameters of 3, 4, and 5 mm and heights of 3, and 4 mm. Sensors were identified by their magnetic element geometry according to the convention: diameter×height-shape, such that a sensor with a cylinder shaped magnetic element that is 2 mm in diameter and 4 mm in height was labeled a 2×4-cylinder sensor.

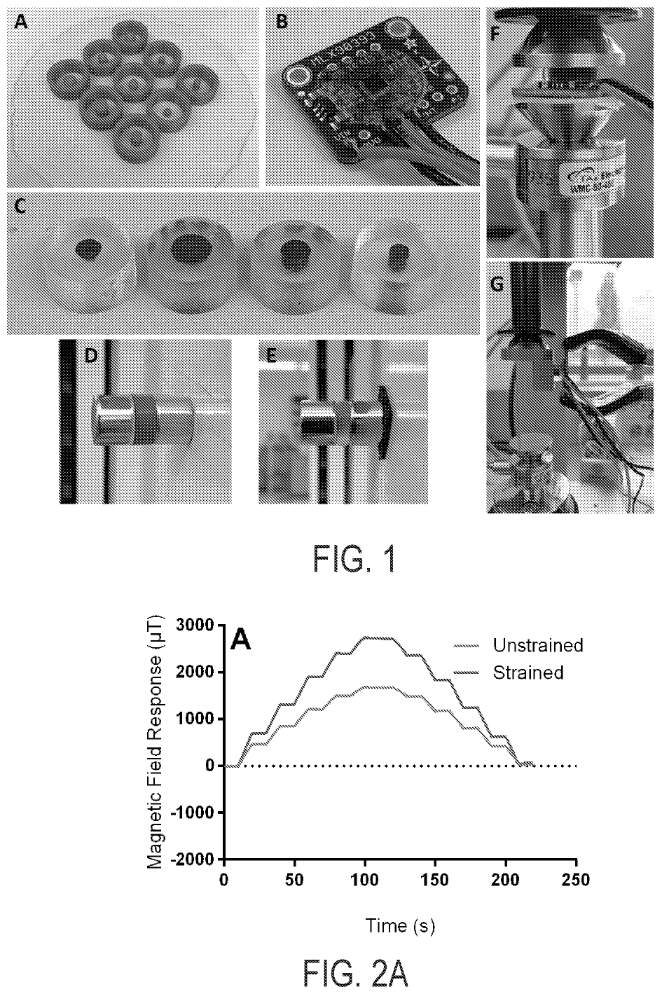

Fabrication was accomplished via a 2-step molding process The mold for the first step was created by 3D printing on a flat plastic surface (FIG. 1A). Each mold included a peg, of dimensions according to the desired magnetic element geometry, to leave a cavity to be filled by the silicone-magnetic powder composite material during step 2 of the process. The liquid silicone precursor was poured into each mold and left to cure in ambient conditions for 48 hours to avoid warping the mold. After curing, the silicone was removed from the mold. Unless otherwise indicated, magnetic powder was mixed with liquid silicone precursor at a 1:1 ratio (50 weight % magnetic filler concentration) and dispensed into the cavity in each of the silicone structures. These silicone cylinders with liquid silicone-magnetic powder composite material were cured at 60° C. for 2 hours to avoid settling of the magnetic powder within the liquid silicone.

### Alignment of Magnetic Dipoles

When creating silicone-magnetic powder composites, it is possible to align the dipoles of the magnetic material while the silicone is curing (Mirzanejad and Agheli 2019; Hellebrekers et al. 2019), or after the silicone has cured (Kim et al. 2018). For this investigation, the dipoles of the magnetic particles were aligned after the silicone was cured to test whether misalignment of particle dipoles under deformation of the sensor corrupts the signal. Compression tests were performed on sensors under two different magnetic dipole alignment schemes. First, the magnetic dipoles were aligned by placing strong permanent magnets (˜650 mT) on both sides of the sensors while the sensor was placed within the outer portion of the original 10 mm by 5 mm cylindrical mold such that the sensor maintained its original shape while under magnetization (FIG. 1D). Using this method, the magnetic particle dipoles would be aligned in the vertical direction while the sensor was not compressed. Thus, deformation of the magnetic element during compression of the sensor would cause misalignment of the particle dipoles. A second magnetic dipole alignment scheme involved placing the sensor between the same strong permanent magnets without the original mold cylinder such that the sensor was strained ˜40% by the force attracting the two magnets (Figure JE). This configuration aligned the magnetic dipoles in the vertical direction while the sensor is compressed. Therefore, the magnetic dipoles would be aligned in the vertical direction upon sensor compression. It is hypothesized that the signal strength would be stronger in the second configuration, because the deformation of the sensor should work to align the dipoles in the vertical direction rather than misalign them.

### Compression and Shear Testing

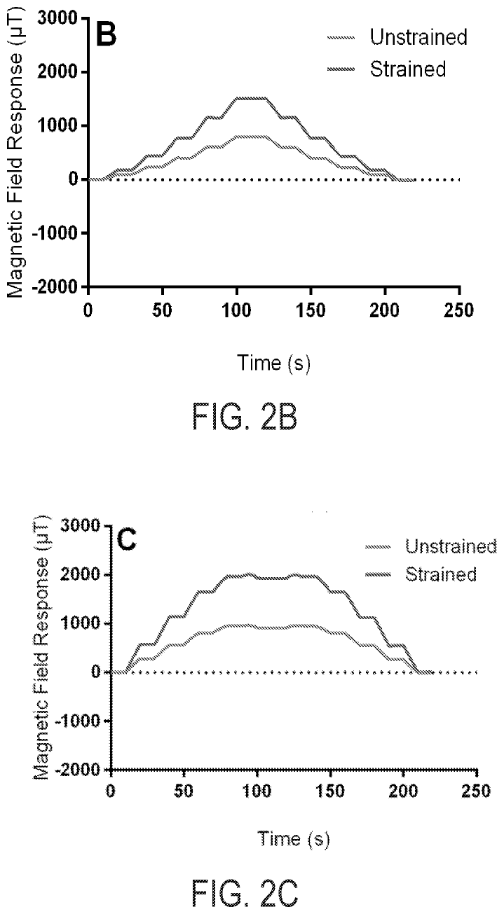

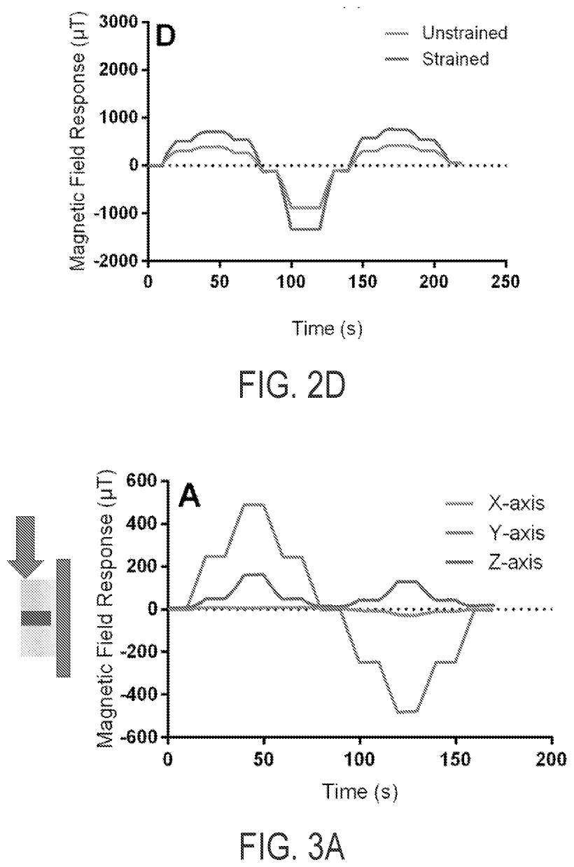

A MLX90393 triple-axis magnetometer breakout board (Adafruit Industries, New York City, NY, USA) was wired to an Arduino Uno microcontroller and communicated data to a computer via I2C. The surface of the breakout board was leveled with the magnetometer chip using polyurethane to avoid unwanted deformation of the sensor constructs around the magnetometer chip (FIG. 1B). The magnetometer board was mounted to the bottom geometry of an Electroforce 5500 (TA Instruments, Eden Prairie, MN, USA) using double-sided tape. The sensors were subsequently placed on the magnetometer and the axial mover of the ElectroForce 5500 was lowered until contact was made with the top side of the sensor. The sensors were oriented with the magnetic element away from the magnetometer giving at least 1 mm of space between the magnetic element and the magnetometer, as pilot studies showed leaving no gap between the magnetic element and the surface of the magnetometer greatly diminishes signal strength. Compressive force was exerted by displacing the axial mover in increments of 0.5 mm up to 2.5 mm of compressive displacement on the samples (FIG. 1F) and returned to 0 displacement in matching decompression steps Each step was held for 10 seconds except the 2.5 mm displacement step was maintained for 20 seconds (FIG. 2).

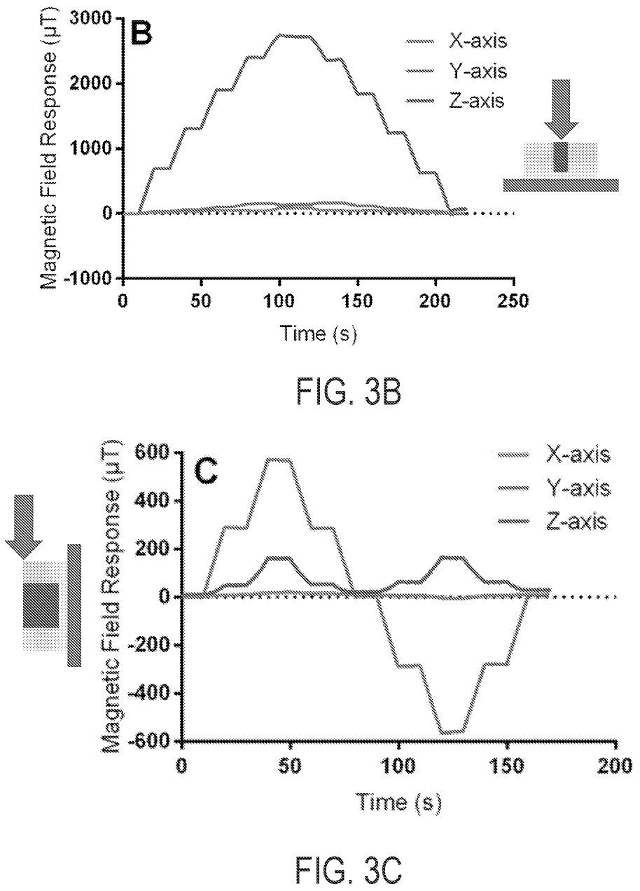

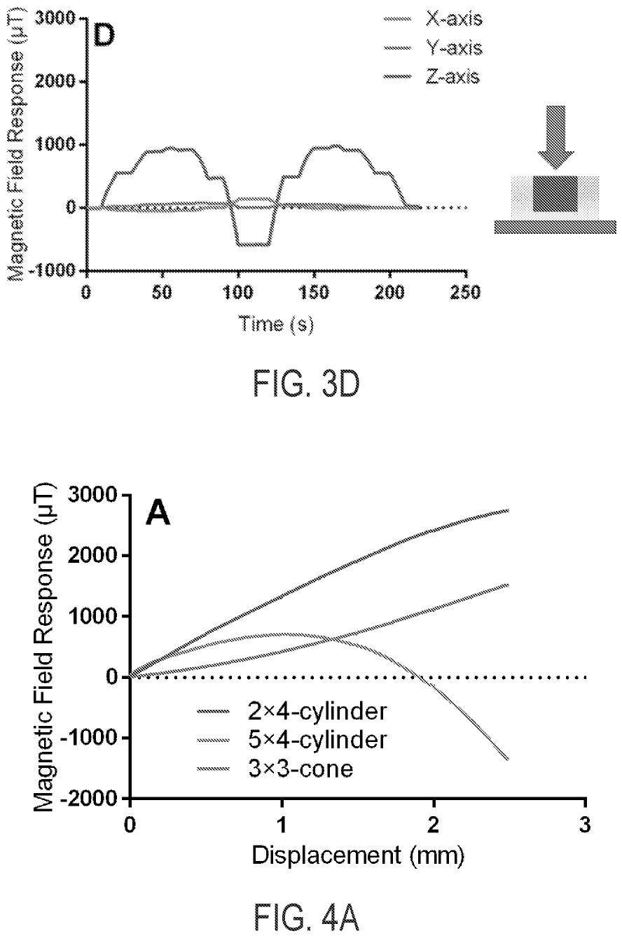

Testing for detection of shear force was also performed with the Electroforce 5500 with an adapted set up (FIG. 1G). The configuration of the sensor construct with respect to the magnetometer was consistent with the compression testing. The top of sensor was pressed against a 3D-printed adapter which was connected to the axial mover of the Electroforce 5500. The axial mover was displaced in 0.5 mm steps for a total of 1 mm displacement in both Y-axis directions. Each step was maintained for 10 seconds (FIG. 3A,C).

Magnetometer readings from all 3 axes, as well as time and axial mover displacement were collected during each test. A load cell attached to the bottom geometry of the Electroforce 5500 collected force data during all compression tests, but was not able to collect force during shear testing.

### Football Helmet Integration and Demonstration

A 2.5×4-cylinder sensor with 80% magnetic filler by weight was integrated into a football helmet (Riddell; Rosemont, IL) by cutting a 10 mm diameter by 5 mm height cavity into the forehead pad of the helmet and securing the sensor in the cavity. A magnetometer was fixed to the sensor and communicated data wirelessly using a WIFI enabled microcontroller module (M5Stick-C, M5Stack, Shenzhen, China) to a nearby computer at a sampling rate of 29 Hz. The in-helmet sensor was calibrated against a 3-dimensional force plate (4060-08, Bertec, Columbus, Ohio, USA), which was mounted vertically and sampled at a rate of 2 kHz, by performing several helmet-force plate impacts from different angles while the helmet was worn. Force data (N) from the force plate were downsampled to match the data from the in-helmet sensor and calibration regression equations were fit to the force plate readings plotted against the raw magnetic field strength readings (μT) from the in-helmet sensor during the impacts. Thirteen experimental trials were performed in which the helmet, while being worn, impacted the vertically mounted 3-dimensional force plate from different angles.

### Statistical Analysis

Magnetic field strength readings (μT) from each axis were normalized by subtracting the average signal recorded during the first 5 seconds of each test when displacement was 0. Several metrics were used when considering the performance of each sensor. The peak magnitude of the sensor was considered the peak observed magnetic field strength reading from the Z-axis of the magnetometer during compression testing, and the average of the absolute values of the peak observed readings from the Y-axis were used in both directions during shear testing. The correlation coefficient of the relationship between the magnetic field response and compressive force applied was considered linearity in terms of force, and the correlation coefficient of the relationship between the magnetic field response and displacement of the axial mover was considered linearity in terms of displacement.

The reliability of the in-helmet sensor in relation to the force plate was assessed by the interclass correlation coefficient (ICC) of the peak force measured in the primary axis of the impact (i.e. z-axis for a straight impact, x-axis for an angled impact) from the in-helmet sensor and the force plate. In addition, the percent error in peak force reading between the in-helmet sensor and the force plate was calculated for each impact.

### Results

### Compression Testing Results

Soft magnetic sensors were constructed using different shapes (cylinders, cones) of PDMS filled with neodymium powder as magnetic elements embedded within larger PDMS cylinder constructs (FIG. 1). These magnetic elements were placed in proximity to a magnetometer to measure changes in magnetic field strength and direction as the sensors were deformed (Table 1). Prior to testing, rare earth magnets were used to magnetize the magnetic powder filler when the magnetic elements were uncompressed or when compressed to ˜40% strain. All sensors were considerably more sensitive when the dipoles of the magnetic particles were aligned while the sensor was under compression. The peak magnitude for each sensor design was increased between 53% and 217% in comparison to tests performed with the magnetic particle dipoles oriented vertically while the sensor was not compressed. Compressing the sensor during magnetic dipole alignment combats an issue previously described where the deformation of the magnetic element causes misalignment of the dipoles with the magnetometer, and consequently a reduction in magnetic field strength (Mirzanejad and Agheli 2019). Therefore, all further results will only be presented from sensors with magnetic particle dipoles aligned while the sensors were compressed.

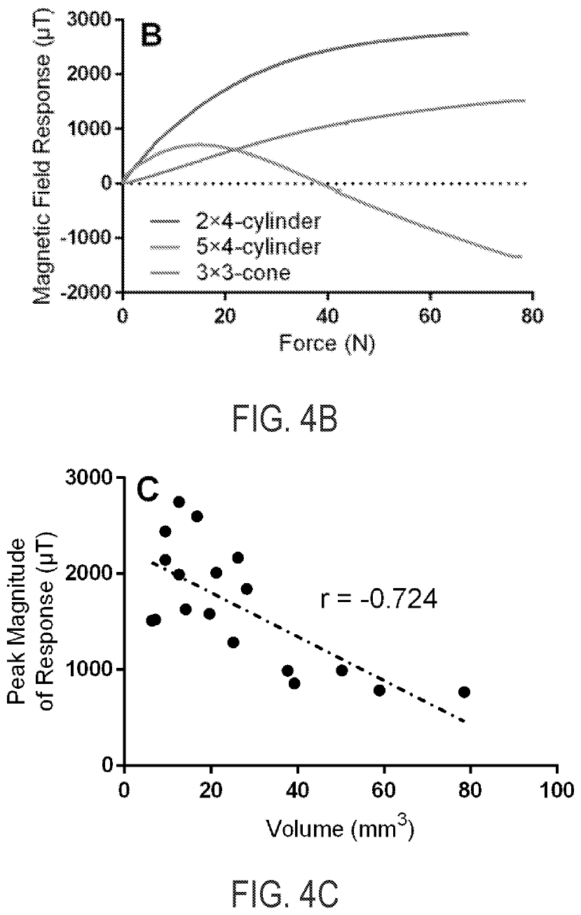

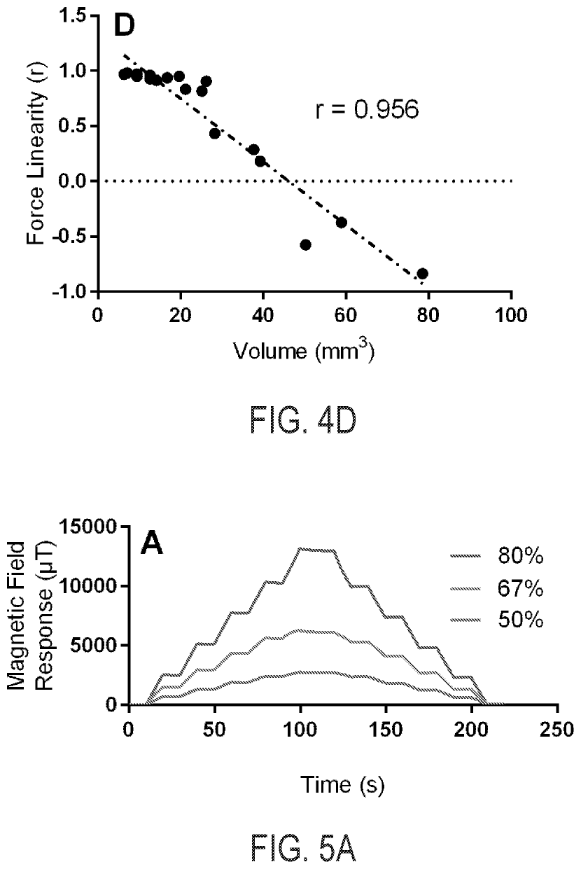

Example compression test data for several sensors with representative magnetic element geometries are presented in FIG. 2. All sensors could detect the rate, duration, and magnitude of compression events to some extent. However, the sensitivity of the response and the linearity of the response in relation to displacement and force varied widely between different magnetic element geometries (Table 1). Sensors with larger volume magnetic elements generally failed at some level of compression, such that further increases in compression resulted in no increase, or the reversal, of the signal (FIG. 4) as previously reported. Each sensor's peak magnitude and the linearity of its magnetic response in relation to force are plotted against the volume of the magnetic element in FIG. 4C-D. These relationships indicated larger magnetic element volumes were actually detrimental to signal quality.

The 2×4-cylinder sensor was the most sensitive design for sensing compression events, as it displayed the largest peak magnitude of 2,748 μT. This sensor also performed well according to all other metrics including the linearity of the response in relation to both displacement and force (Table 1). The sensor designed with the largest magnetic element, the 5×4-cylinder sensor, paradoxically had the smallest peak magnitude of all sensors (766 μT) and performed poorly by all other metrics considered. This sensor was only able to sense compression up to ˜1 mm (20% strain) before further compression resulted in a reversal in the signal direction (FIG. 2D).

### Shear Testing Results

Example shear displacement test data for representative magnetic element geometries are presented in FIG. 3A and C. Table 1 displays the peak response from the Y-axis averaged across the two directions of shear displacement. All sensors adequately detected the rate, duration, and magnitude of shear displacement events and no sensor design presented the signal quality issues, which have been discussed for larger magnetic element volumes during sensing of compression events. The linearity of the response in terms shear displacement was greater than r=0.998 for all sensor designs.

The trend for poorer performance from larger magnetic element volume sensors did not apply to sensing shear forces. The sensor with a 4 mm diameter by 4 mm height cylinder magnetic element was the most sensitive design with a peak magnitude of 570 μT. In addition, all cylinder magnetic elements with 4 mm height were more sensitive than all other sensor designs for sensing shear displacement. The 2 mm by 4 mm cylinder magnetic element performs well in shear and normal compressive dimensions. The 4 mm by 4 mm cylinder magnetic element has marginally greater sensitivity for shear displacement, but poor quality for normal compression sensing.

### Magnet Diameter, Magnetic Filler Concentration, and Magnet Location

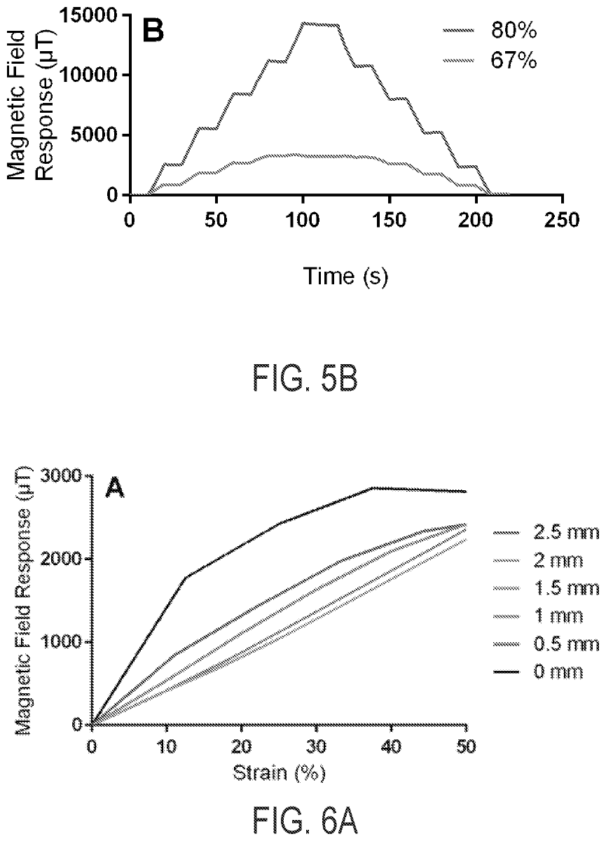

To investigate optimal magnetic element geometry in finer resolution, and the effect of increasing magnetic filler concentration on sensor sensitivity, additional sensors were constructed. Each sensor's magnetic element was 4 mm in height, with diameters ranging from 1 to 3 mm (in increments of 0.5 mm) and with magnetic filler concentrations for the magnetic element of 67 and 80 weight %. As expected, signal strength was increased significantly in sensors with magnetic elements with higher magnetic filler concentration. The 2×4-cylinder sensors with 67% and 80% magnetic filler concentration showed 2.3 and 4.8 fold increases, respectively, in sensitivity over the same sensor design with 50% magnetic filler concentration (FIG. 5) of the magnetic element.

The 2×4-cylinder sensors outperformed the other magnetic element diameters tested for sensors with 67% magnetic filler concentration, which agrees with the results from the original study in sensors with 50% magnetic filler concentration. The 1×4-cylinder and 1.5×4-cylinder sensors were not as sensitive as the 2×4-cylinder sensor, and the 2.5×4- cylinder and 3×4-cylinder sensors displayed the typical signal quality issues previously described for sensors with larger magnetic element diameters. For the sensors with 80% magnetic filler concentration, the 2.5×4-cylinder sensor displayed the highest peak magnitude and did not have the signal quality issues present in the 2.5×4-cylinder sensor with 67% magnetic filler concentration. Thus, increasing the magnetic filler concentration may also increase the maximum diameter of magnetic element that can be compressed to 50% strain without displaying a blunted or reversed signal response. This is likely due to the higher concentration of magnetic filler stiffening the material, such that less deformation of the magnetic element occurs during compression. Indeed, photogrammetry indicated the increase in diameter of the magnetic element of sensors composed of 80% magnetic filler was less than those for sensors with 50% magnetic filler concentration. At a 50% strain, the diameters of the magnetic elements of the 2×4-cylinder sensors were increased by 70.6% and 55.8% for magnetic elements composed of 50% and 80% magnetic filler respectively. Likewise, the diameters of the magnetic elements of the 3×4-cylinder sensors were increased by 47.6% and 41.7% for magnetic elements composed of 50% and 80% magnetic filler respectively.

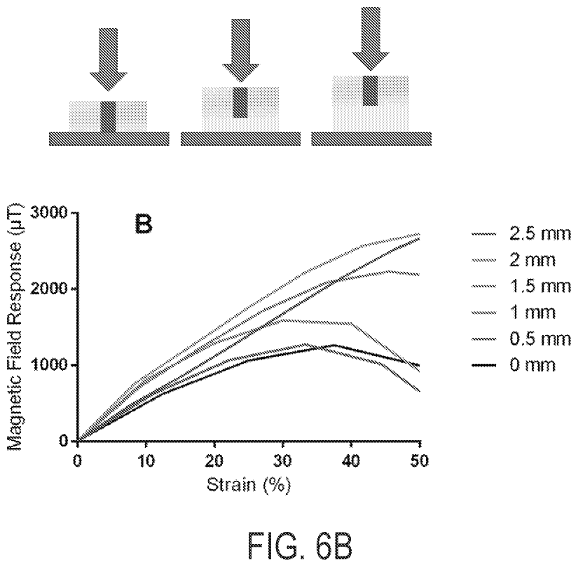

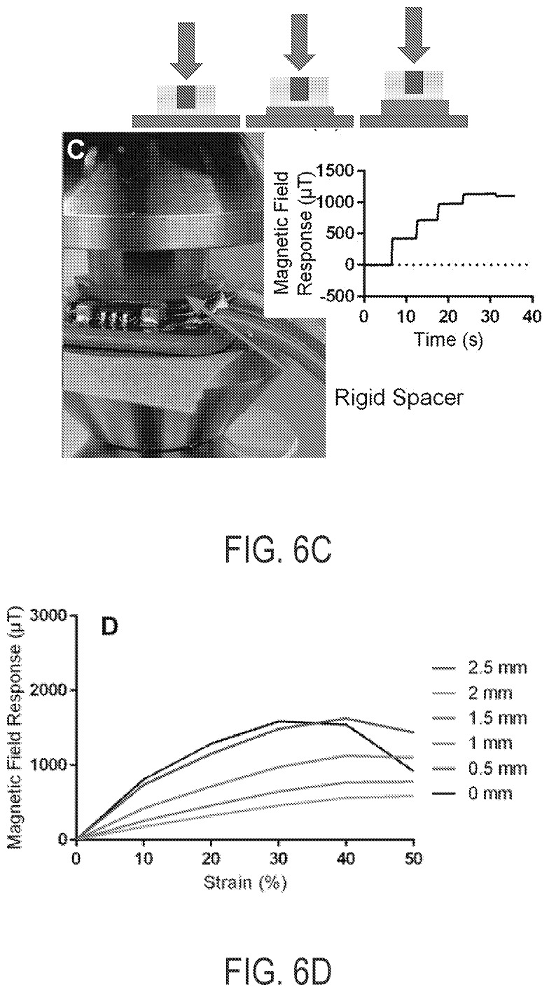

To investigate the optimal sensor height while holding the magnetic element geometry constant, 2×4-cylinder and 3×4-cylinder sensors, with 50% magnetic filler concentration for the magnetic element, were constructed with a total sensor height of 4 mm, such that the magnetic element was exposed on the bottom of the sensor and would be in direct contact with the magnetometer Pure silicone discs of varying height, but the same material (i.e. same stiffness) and diameter of the sensors were created such that they could be added between the magnetometer and the sensor as spacers to increase the total sensor height. Testing was performed by compressing each sensor configuration to 50% strain in 0.5 and 0.25 mm steps. For the 2×4-cylinder sensor, as the overall height of the sensor increases, so does the linearity of the signal (in relation to strain), but the sensitivity of the signal is slightly decreased concomitantly (FIG. 6A). For the 3×4-cylinder, the linearity of the signal increases drastically as the height of the sensor is increased, and the sensitivity of the sensor also increased until the overall height of the sensor exceeds 6 mm (FIG. 6B). Therefore, the optimal sensor height varies by the geometry of the magnetic element as larger magnetic elements require taller sensors, which increase the space between the magnetic element and the magnetometer, in order to function properly. However, taller overall sensors may be inappropriate for many wearable technology applications, which require a low-profile sensor.

Similar testing was performed in an experiment to quantify the effect of increasing the remote distance between the silicone-magnetic powder sensor construct and the magnetometer. In this experiment, rigid (polylactic acid) plastic discs were 3D printed and used as spacers between the magnetometer and the sensor to simulate remote sensing at different distances (FIG. 6C). All testing for this experiment was performed on a 3 ×4-cylinder sensor of the same design as in the original study described in the methods (5 mm total height). Increasing the remote distance between the magnetometer and the sensor did increase the linearity of the signal such that the signal continued to show increases in magnetic field strength up to a strain of 50% when the distance was 1.5 mm or greater; however, the sensitivity of the signal was reduced (FIG. 6D). Increasing gap (or sensor height) can make larger sensor geometry signals more linear.

### Football Helmet Demonstration

American football has been associated with a greater number of concussions than any other sport currently played in the United States (Daneshvar et al. 2011), and understanding kinetic characteristics of head collisions may be a vital factor in understanding, preventing, and offering an appropriate treatment to concussed individuals (McAllister and McCrea 2017). To assess the utility of the current sensor design in a real-world application, a 2.5×4-cylinder sensor with 80% magnetic filler concentration was integrated into an existing pad within a football helmet. The additional padding in the helmet rendered this sensor imperceivable to the wearer, thus allowing studies of helmet impacts using a traditional force place on the impact surface compared to a magnetic sensor within the helmet.

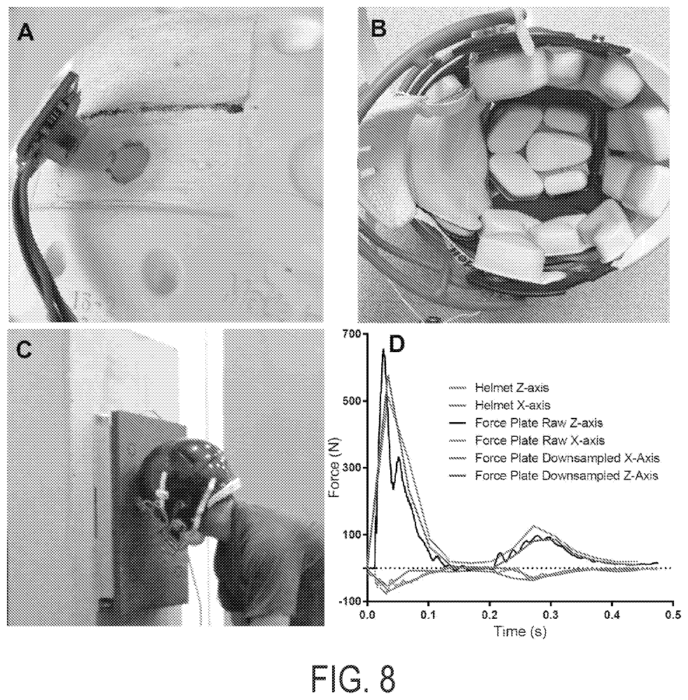

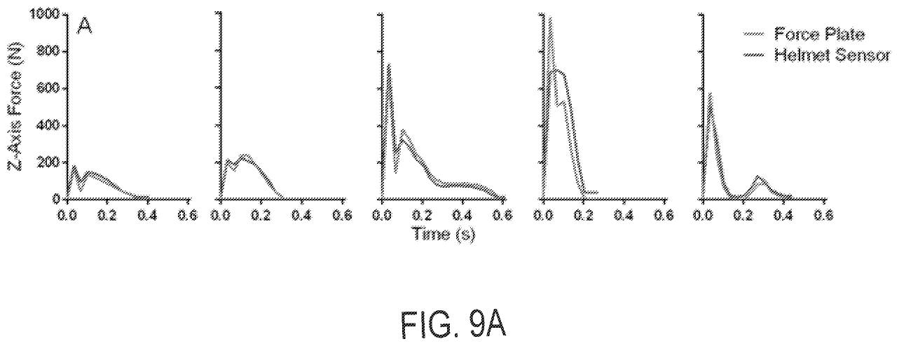

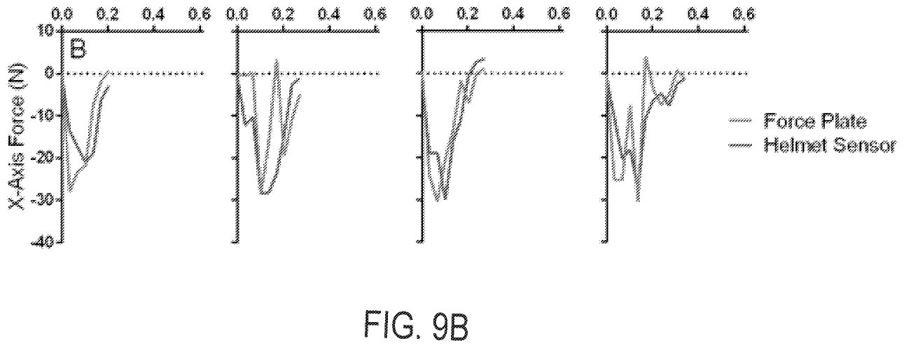

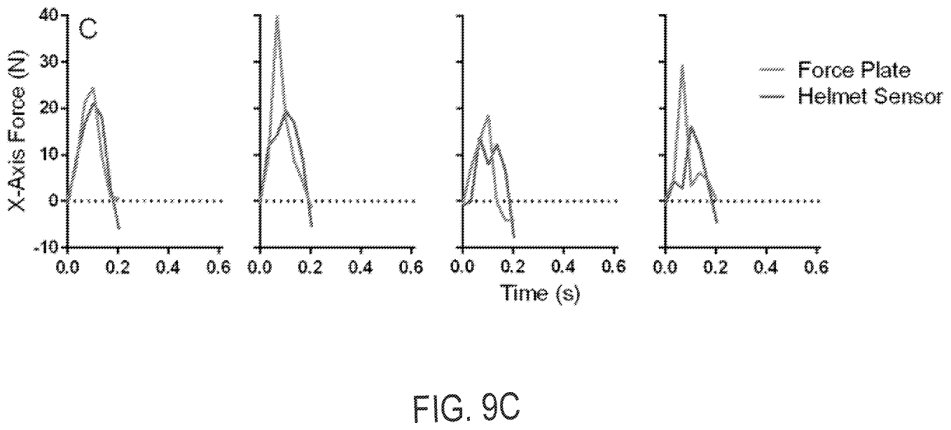

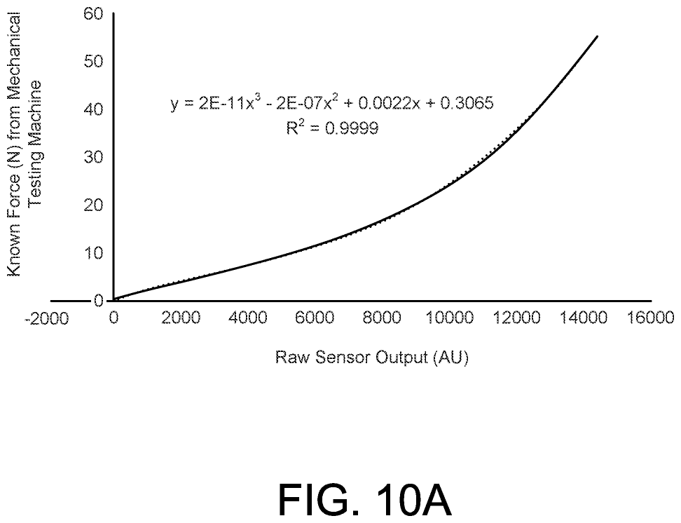

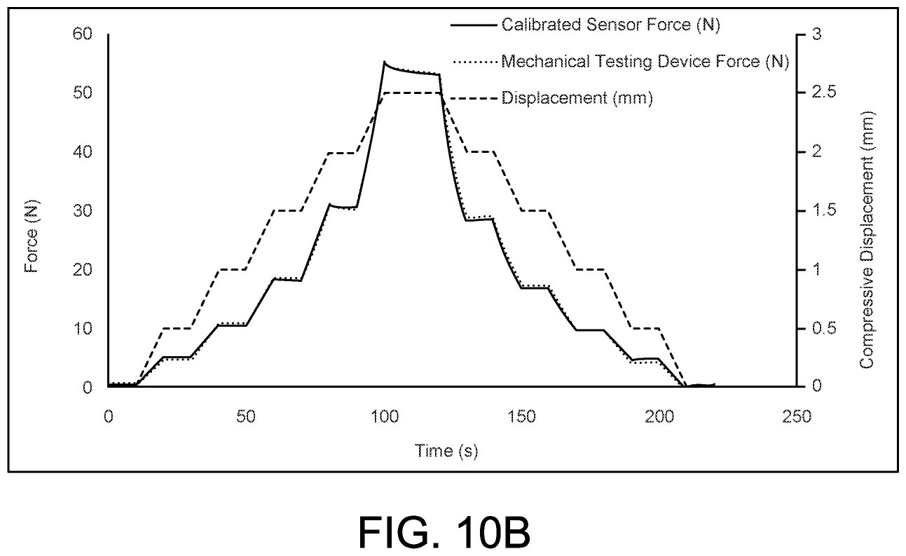

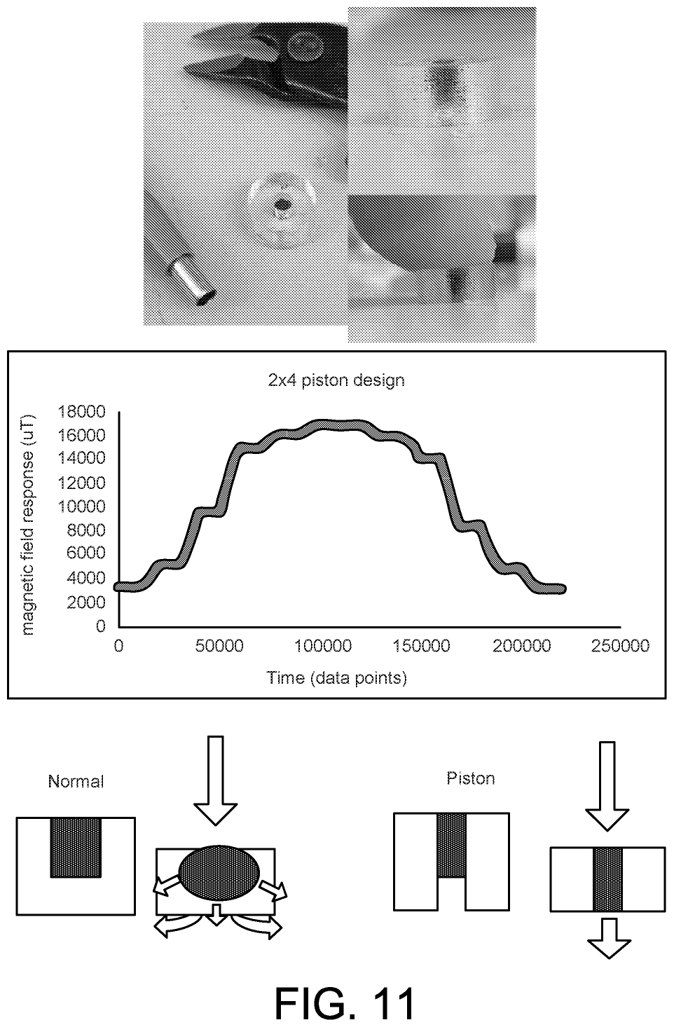

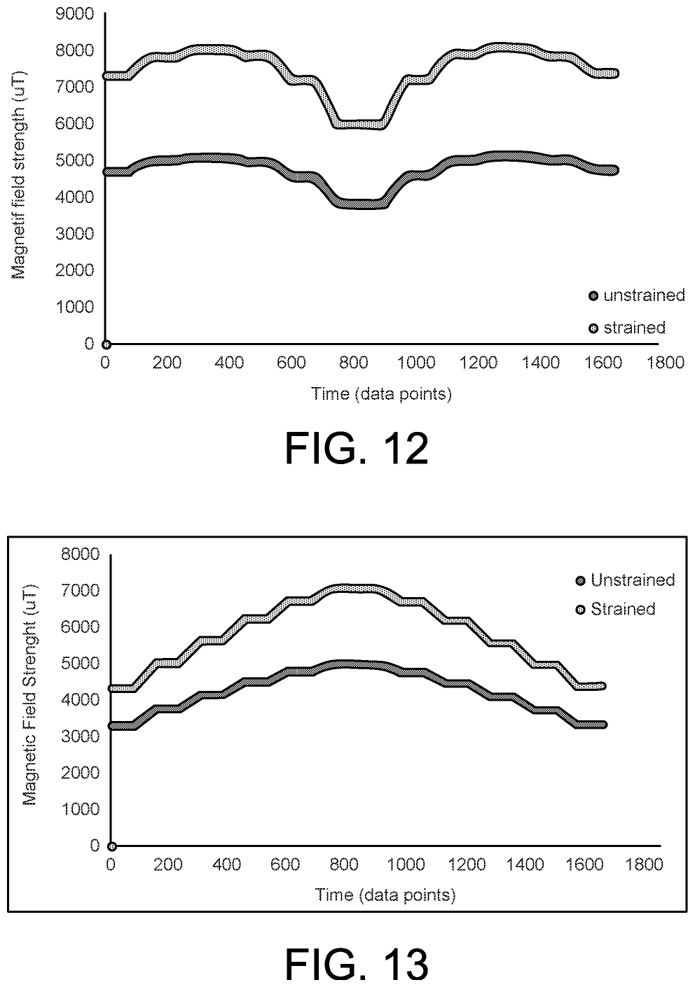

Force vectors were clearly observable for all trials by analyzing the force-time curves of each of the three axes of the in-helmet magnetic sensor (FIG. 8A-B). The sensor displayed excellent agreement with the force plate for peak force (ICC=0.981). In addition, peak forces for 8 of 13 impacts exhibited moderately low error (1-13%) when compared to the force plate, but the several impacts had larger errors (25-51%). The sensor had high overall agreement with the force plate according to the ICC, and most impacts were accurately measured; however, a higher sampling rate is likely needed for consistent and accurate quantification of impacts which contain high frequency components. Data from all helmet-force plate impacts are presented in FIG. 9.

### Discussion

### Signal Quality for Composite Sensor Designs

Many previous researchers who have designed sensors employing magnets embedded within PDMS or other soft materials have detailed the mechanisms by which the displacement of the magnetic element is used to sense applied forces (Wang et al. 2016b, b; Rosle et al. 2019), including sensors designed with silicone-magnetic powder composites (Mirzanejad and Agheli 2019; Hellebrekers et al. 2019). Because silicone-magnetic powder composites are subject to deformation unlike rigid permanent magnets, the magnetic field can deviate during deformation. The implications of this fact in regards to soft sensor applications have been modeled and discussed previously (Mirzanejad and Agheli 2019). The authors showed that the magnetic dipoles of the particles within the composite magnetic element deviated from the vertical direction increasingly under compression, which contributed to the observed reversal of the direction of the signal at certain levels of force (varying depending on sensor design), such that sensors could only be operated below certain levels of force or strain. In the present study, large increases in signal strength were observed for each sensor design when the dipoles of the magnetic particles were aligned while the sensor was compressed. Thus, increasing compression aligned the magnetic dipoles with the Z-axis of the magnetometer, rather than shifted dipoles away from alignment with the Z-axis of the magnetometer. This finding confirms shifting the alignment of the magnetic dipoles must be considered in compression sensors using silicone-magnetic powder composites.

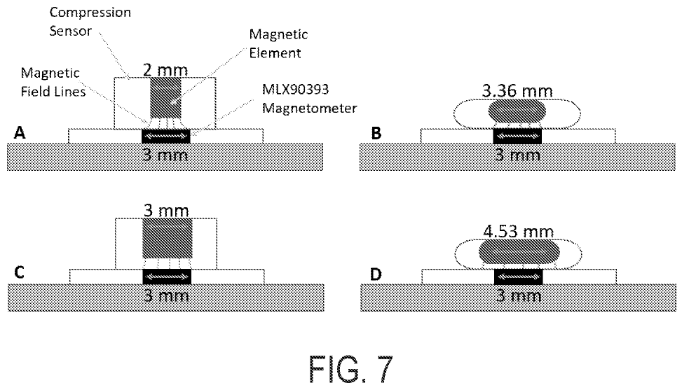

Surprisingly, the blunting or reversal of the signal direction beyond certain levels of strain was persistent regardless of magnetic dipole alignment, but this phenomenon was limited to larger volume magnetic elements, which invites additional consideration of the signal quality issues. This second injury to signal quality was the lateral displacement of magnetic particles during compression due to the deformation of the magnetic element (FIG. 7). MLX90393 magnetometers have a 3 mm×3 mm square surface, thus it is plausible lateral movement of magnetic particles toward or beyond the border of the magnetometer surface may act to decrease the magnetic field strength reading. For cylinder magnetic elements in the present study, magnetic elements with a diameter of 2 mm or less (<67% of the width of the magnetometer) exhibited an increased signal with increasing deformation up to a strain of 50%. Photogrammetry indicated the diameter of the 2×4-cylinder sensor's magnetic element was increased to 3.36 mm at 50% strain, which is slightly larger than the width of the magnetometer, and sensors of this design did not display major signal quality issues. However, the signal for 3×4-cylinder sensors was reversed at strains >40% such that further compression caused a reduction in magnetic field strength. Photogrammetry indicated the diameter of the 3×4-cylinder sensor's magnetic element was increased to >4 mm at strains above 40%. Therefore, the compression caused the magnetic particles on the periphery of the magnetic element to be displaced >1 mm laterally from the z-axis of the magnetometer, with their resulting position being >1 mm lateral to the border of the magnetometer. Such particles are acting in opposition to the increase in magnetic field strength, which becomes the dominant signal (reversing the overall signal) at higher strains for sensors containing larger magnetic elements. A single exception to this rule was the 2.5 mm diameter sensor (83% of the width of the magnetometer) with 80% magnetic filler concentration which displayed a linear increase in magnetic field strength up to 50% strain. This is likely due to its stiffer magnetic element and photogrammetry confirmed less deformation of magnetic elements with 80% magnetic filler concentration. Overall, the geometry of the magnetic element and its position with respect to the magnetometer primarily effects the linearity of the signal with compression, while the quantity of magnetic material primarily effects the sensitivity or strength of the signal.

### Capacity for Sensing Shear Forces

The capacity for soft force sensors to accurately quantify shear forces or displacement would also advance the field and broaden applications for monitoring biomechanics. For example, shear forces acting upon foot soles are important risk factors for development of foot ulcers in patients with diabetic neuropathy (Wu et al. 2007; Lam et al. 2017), and the detection of ground reaction forces in horizontal planes are desirable for biomechanical analysis of sport movements (Holm et al. 2008). Finally, no true 3-dimensional force sensors are currently available for instrumented football helmets (Siegmund et al. 2016; Merrell et al. 2017), although such technology could aid in the proper analysis of head impacts for combatting the prevalence of traumatic brain injuries.

Signal quality issues were not as evident when sensing shear in controlled experiments as all sensors had very linear responses during shear displacement when compared to compression sensing. All sensors also possessed specificity in the signals of each axis such that vertical and horizontal forces could be easily distinguished during both shear displacement (FIG. 3A,C) and during normal compression tests (FIG. 3B,D). It appears the most important sensor design specification for sensitivity in the shear directions was height of the magnetic element. The 4×4-cylinder sensor displayed the greatest peak magnitude of 570.7 μT. However, the 2×4-cylinder sensor displayed the greatest peak magnitude for shear sensing (487.4 μT) when comparing only the sensors which displayed no major signal quality issues during compression testing.

### Considerations for Wearable Applications

No sensor design was superior by all metrics, therefore the most effective design could be optimized for the specific application using parameters investigated here. For example, the 3×3-cone sensor displayed the most linear magnetic field response in relation to force (r=0.980) of all sensor designs and required the second least magnetic material (Table 1). The sensitivity and linearity of larger magnetic elements, such as the 3×4-cylinder sensor, were increased with taller overall sensor designs; however, this increase in height may render the sensor inappropriate for many wearable applications (FIG. 6B). The 2.5×4-cylinder sensor with 80% magnetic filler concentration displayed the highest sensitivity of the sensors, which were capable of sensing force up to 50% strain (FIG. 5B), although the magnetic element was stiff enough to be potentially perceivable to the touch if compressed firmly. However, for many applications, such as for the football helmet presented here, this does not negatively affect the efficacy of the sensor. Several millimeters of additional padding separated the wearer's head and the sensor which made the sensor imperceivable, yet force was still transmitted through the additional padding such that the sensor can function properly.

### Conclusion

Previous designs for compression sensors using a magnetometer to detect the displacement of a rigid magnet embedded in silicone, or a silicone-magnetic powder composite, have had feasibility, functionality, and signal quality limitations, which have restricted their utility. Several experiments were performed to identify design parameters to improve sensor sensitivity and accuracy and consequently broaden the applications of these sensors to include wearable technology. Sensors composed of a silicone-magnetic powder composite provided a platform for designing low-profile sensors without saturating the magnetometer while remaining soft enough for potential use in wearable technologies. Magnetizing the magnetic element of the sensor while the sensor is compressed to align the dipoles of the magnetic particles reduced unwanted deviation of the magnetic field during deformation and thereby improved signal fidelity. Lastly, the diameter of the magnetic element was controlled, in relation to the width of the magnetometer, to ensure the lateral movement of magnetic particles during deformation of the sensor did not corrupt the signal. Sensors fabricated according to these design specifications were able to detect the rate, duration, and magnitude of force in 3 dimensions with highly linear signals and performed properly in detecting head impacts when integrated into a football helmet.

Additionally, the magnetic element should be as tall as possible (within overall sensor size constraints) but should be at least 1 mm gap between magnetic element and magnetometer if 50% strain is needed. If lower levels of strain are expected to be the maximum, this gap can be reduced for increased sensitivity with small diameter magnetic elements.

### REFERENCES

Biswas S, Shao Y, Hachisu T, et al (2020) Integrated Soft Optoelectronics for Wearable Health Monitoring. Adv Mater Technol 5:2000347.
Daneshvar D H, Nowinski C J, McKee A C, Cantu RC (2011) The Epidemiology of Sport-Related Concussion. Clinics in Sports Medicine 30:1-17.
Hellebrekers T, Kroemer O, Majidi C (2019) Soft Magnetic Skin for Continuous Deformation Sensing. Advanced Intelligent Systems 1:1900025.
Holm D J, Stålbom M, Keogh J W L, Cronin J (2008) Relationship Between the Kinetics and Kinematics of a Unilateral Horizontal Drop Jump to Sprint Performance: Journal of Strength and Conditioning Research 22:1589-1596.
Johnson J G, Ozkan S S Three Dimensional Force Sensing Array with Applications in Robotics and Biomechanics. 76
Kim Y, Yuk H, Zhao R, et al (2018) Printing ferromagnetic domains for untethered fast-transforming soft materials. Nature 558:274-279.
Lam B L, Stomberg N A, Laovoravit T D (2017) MagneForce: Validation of a Modular Tri-Axial Force Sensor for Gait Analysis. 112
Low J H, Khin P M, Yeow C H (2015) A pressure-redistributing insole using soft sensors and actuators. In: 2015 IEEE International Conference on Robotics and Automation (ICRA). IEEE, Seattle, WA, USA, pp 2926-2930.
McAllister T, McCrea M (2017) Long-Term Cognitive and Neuropsychiatric Consequences of Repetitive Concussion and Head-Impact Exposure. Journal of Athletic Training 52:309-317
Merrell A J, Christensen W F, Seeley M K, et al (2017) Nano-Composite Foam Sensor System in Football Helmets. Ann Biomed Eng 45:2742-2749.
Mirzanejad H, Agheli M (2019) Soft force sensor made of magnetic powder blended with silicone rubber. Sensors and Actuators A: Physical 293:108-118.
Rosle, Wang, Hirai (2019) Geometry Optimisation of a Hall-Effect-Based Soft Fingertip for Estimating Orientation of Thin Rectangular Objects. Sensors 19:4056.
Siegmund G P, Guskiewicz K M, Marshall S W, et al (2016) Laboratory Validation of Two Wearable Sensor Systems for Measuring Head Impact Severity in Football Players. Ann Biomed Eng 44:1257-1274.
Wang H, de Boer G, Kow J, et al (2016a) Design Methodology for Magnetic Field-Based Soft Tri-Axis Tactile Sensors. Sensors 16:1356. https://doi.org/10.3390/s16091356
Wang H, de Boer G, Kow J, et al (2016b) A Low-cost Soft Tactile Sensing Array Using 3D Hall Sensors Procedia Engineering 168:650-653.
Wu S C, Driver V R, Wrobel J S, Armstrong D G (2007) Foot ulcers in the diabetic patient, prevention and treatment. Vasc. Health Risk Manag. 3:65-76

### Example 2: Magnetic Cushion and Remote Sensing

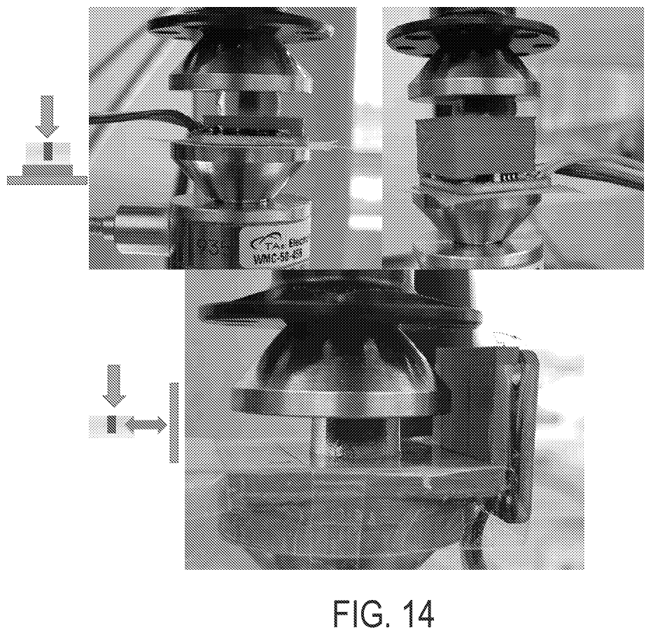

Different vertical and horizontal remote distances were tested when force was applied on vertical and horizontal orientations (see FIG. 14).

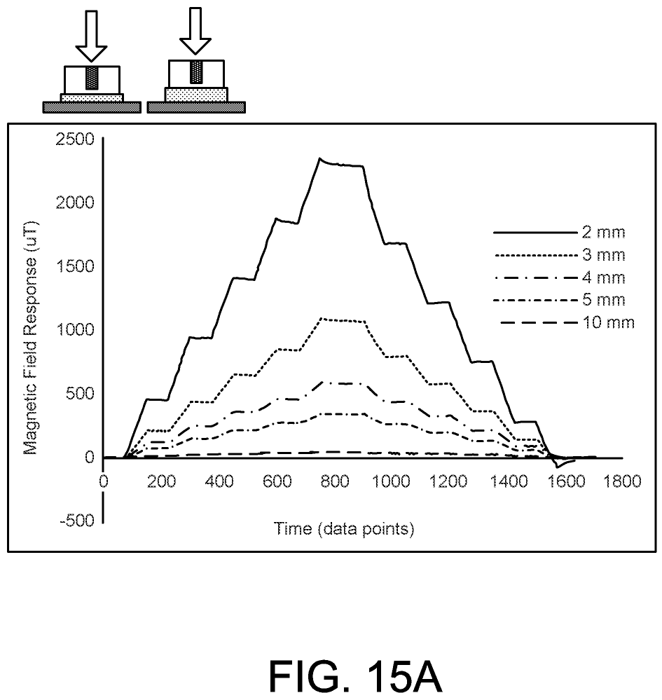

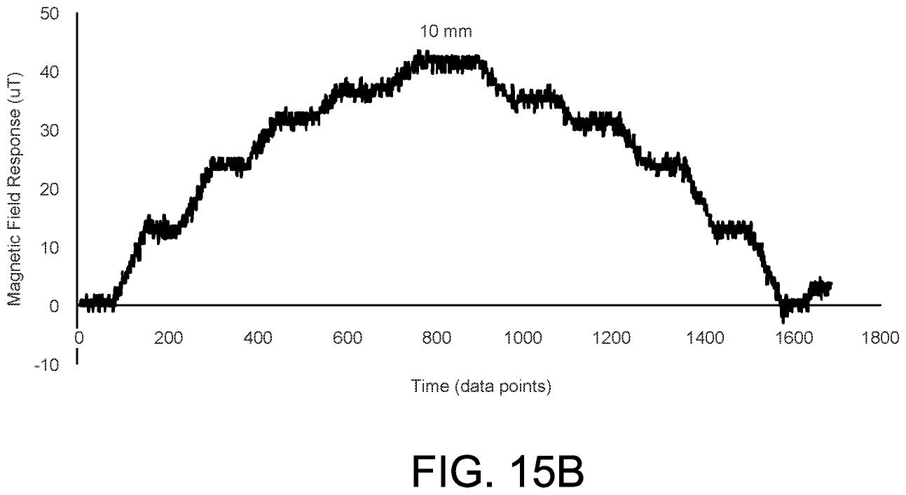

Using a 10×5 mm cylinder cushion with a 2.5×4 cylinder magnetic element (80% Nd) different vertical remote distances were tested from 2 mm to 10 mm. Signal decrease with distance (see FIG. 15).

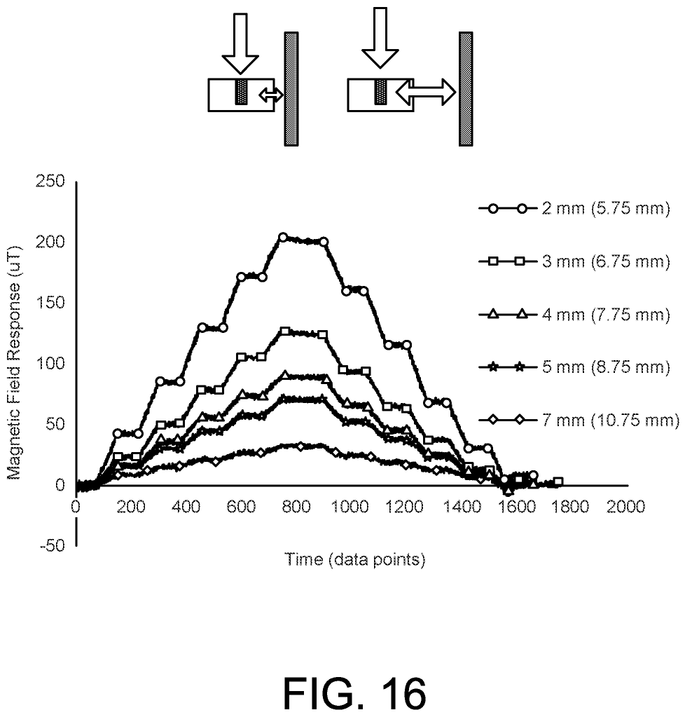

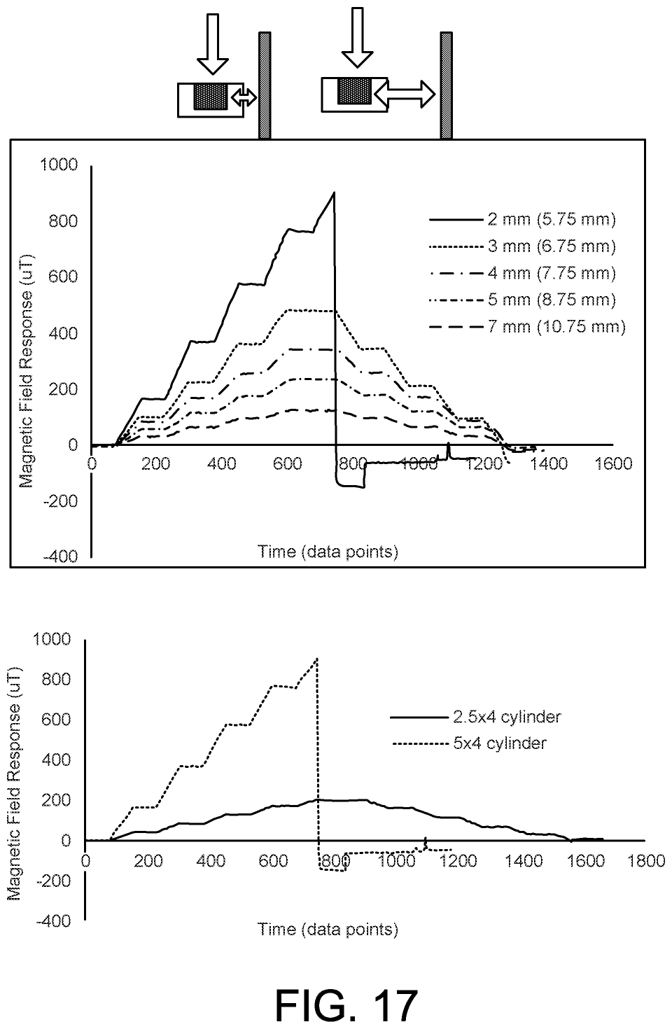

Using a 2.5×4 mm or 5×4 mm cylinder, 80% Nd magnetic element different horizontal remote distances were tested for gap from rubber cushion to magnetometer board 2-7 mm (true gap from magnetometer to magnetic element 5.75-10.75 mm) (see FIG. 16 and FIG. 17). When using a 5×4 mm cylinder, 80% Nd magnetic element 50% strain reached using electroforce (150 N limit reached).

A strong signal at approximately 5 mm distance was measured and measurable signal remains at approximately 10 mm distance.

### Example 3: Piston Magnetic Cushion and Remote Sensing

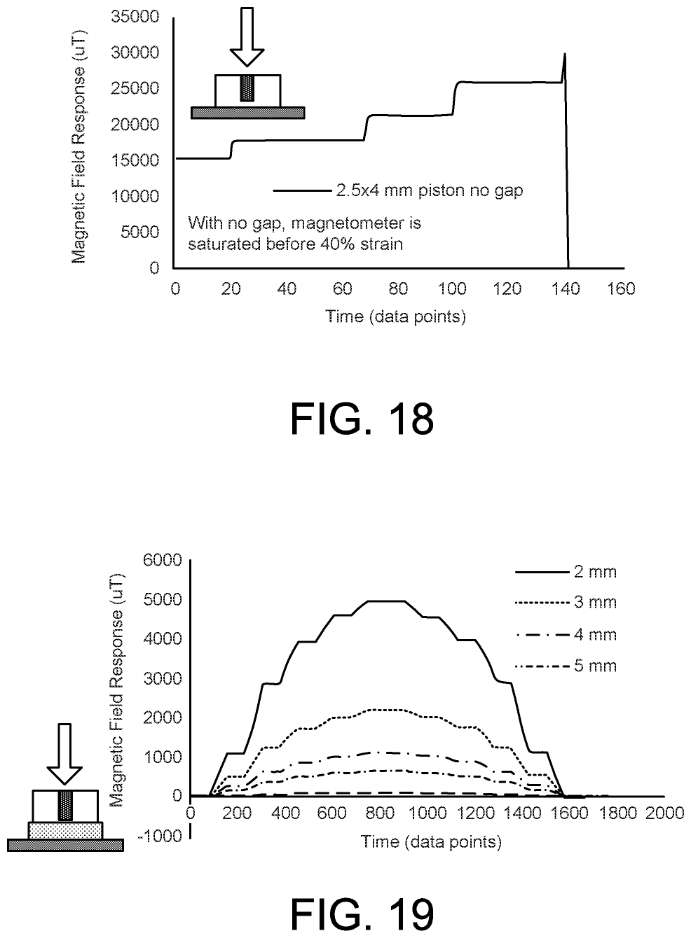

Different vertical remote distances were tested from 2 mm to 10 mm when force was applied on vertical orientations using a piston design setup with a 10×5 mm cylinder cushion and a 2.5×4 cylinder magnetic element (80% Nd) (see FIG. 18 and FIG. 19). Piston design displayed an overall sensitivity increase of approximately 2×compared to non piston designed. However, using a 2.5×4 mm, 80% Nd magnetic element in a 5 mm tall sensor saturates magnetometer before 40% strain is reached.

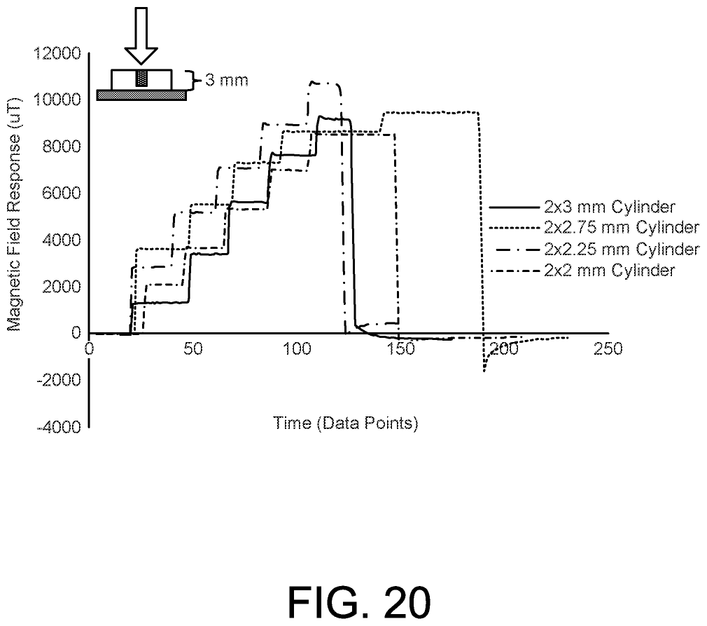

Sensors with a shorter height were tested such as sensor with a 3 mm total height rubber cushion and a 2 mm diameter by 2-3 mm height cylinder magnetic element. 1 mm of compression in 0.2 mm steps (33% strain) was applied to each sample tested. The 3 mm total height sensors showed high sensitivity as shown in FIG. 20. The 2×3 mm cylinder magnetic element with no gap between the magnetic element and magnetometer showed sensitivity up to 33% strain. 2×2.25 mm cylinder with a 0.75 mm gap was the most sensitive at 33% strain (see FIG. 20).

### Example 4: 3D-Printed Magnetic Sensor Cushions

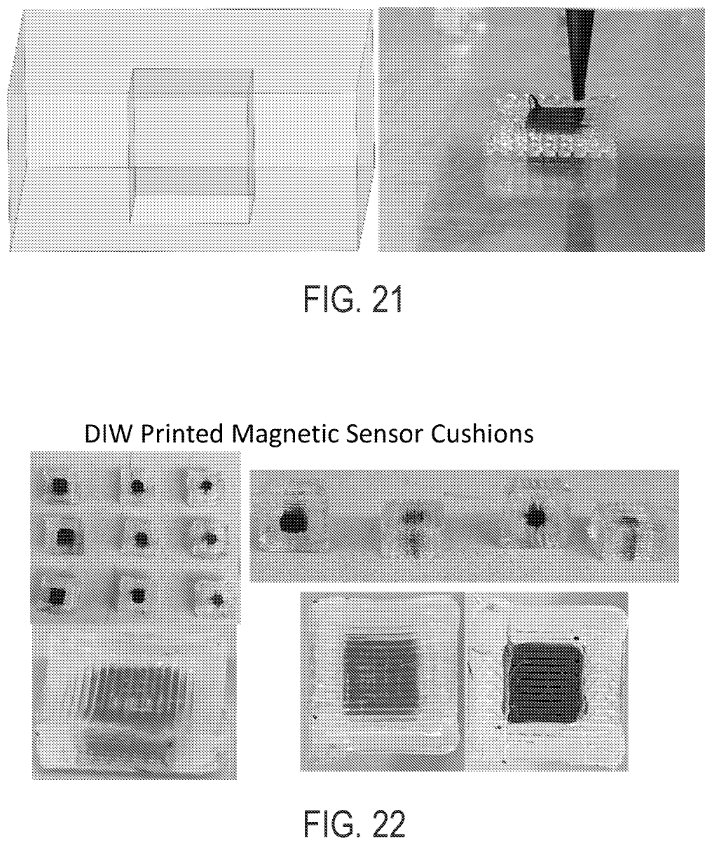

A 10.5×10.5×5 mm construct with variable size magnetic elements was printed (FIG. 21). The materials used were nonmagnetic ink SE 1700, and magnetic ink SE1700 with 25% wrt weight NdFeB microparticles. The infill had 0.5 mm strand spacing.

Printing parameters are shown in Table 2 below.

Magnetic Printing Parameters

| | | |
|---|---|---|
| Pressure | 2.8 | bar |
| Pre-Flow | 0.04 | s |
| Post-Flow | −0.38 | s |
| Speed | 4.1 | mm/s |

Samples can include a 5 mm tall cushion with a 4 mm magnetic portion which is printed first followed by a 1 mm SE1700 layer printed on top (see FIG. 22).

For the 2 dual designs a 0.65 mm offset between was used to allow for full integration of the magnetic and nonmagnetic portion without any unnecessary overlap that can cause smearing and lead to magnetic defects placed throughout the nonmagnetic portion. Some important printing parameters include a couple tenths of a bar increase in pressure due to incorporation of magnetic filler.

FIG. 22 show the prints of different dimensions including 4×4, 3×4, and 2.4×4.

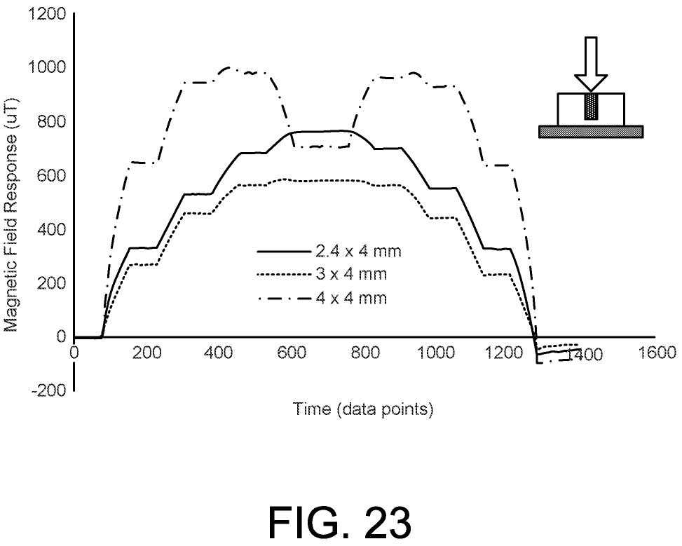

Printed magnetic sensor cushions performance is shown in FIG. 23. The 4×4 mm rectangular prism has higher initial sensitivity. The 2.4×4 mm rectangular prism has most linear signal up to 40% strain.

### Example 5: Flat Flex Circuit Sensors

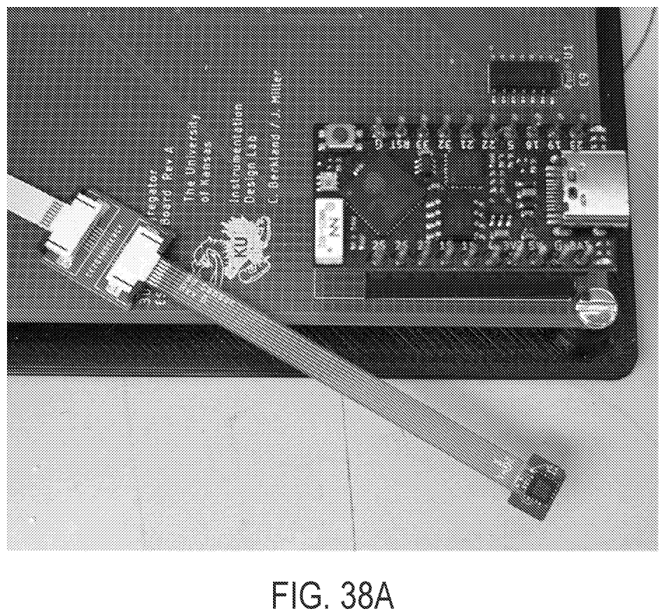

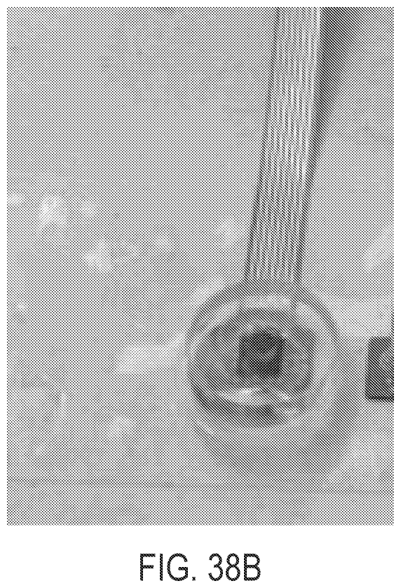

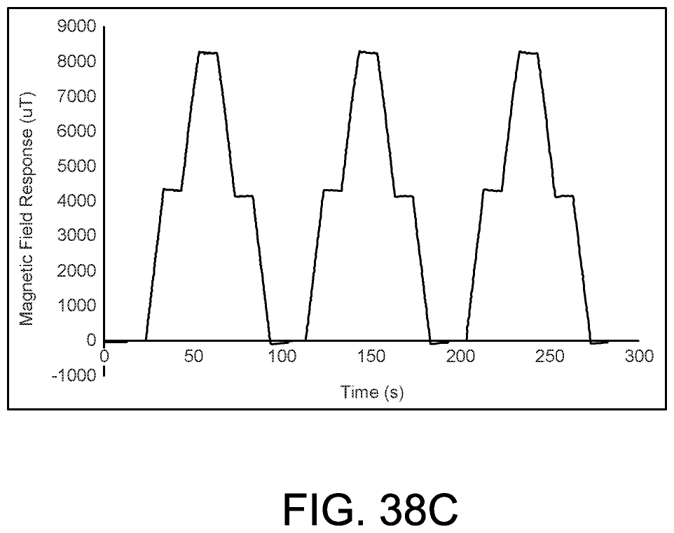

In this Example, we demonstrate that the sensors described herein can also be accomplished using magnetometers mounted on flat flexible circuits as shown in FIGS. 38A-38C. This strategy allows for the fabrication of sensors without the use of a rigid circuit board. As a consequence, the entire sensor can be relatively compliant, making the sensor more suitable for use in wearable technology applications. The signal from circuits made with flat flex cables is robust and appears to be similar in quality to sensors made with magnetometers mounted on rigid circuit boards

### Demonstration of Sensor Units Bonded With Different Adhesives

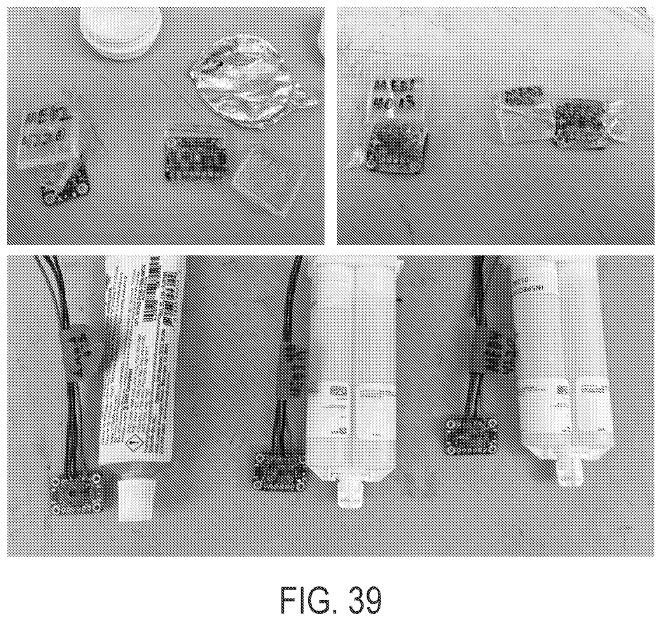

A selection of silicone-based adhesives were screened use in bonding the rubber components of the sensors to the magnetometers (FIG. 39). Four silicone-based adhesives made by NuSil (MED1-4013, MED3-4013, MED2-4220, and MED4-4220) and one made by Smooth-On (Silpoxy) were tested qualitatively to determine whether they could sufficiently bond to silicone and the magnetometer boards with sufficient strength for a functional force sensor. Silpoxy, MED4-4220 and MED3-4013 appeared to have sufficient bonding capabilities with the desired materials, and thus were used for the subsequent sensor embodiments.

### Sensor Construction

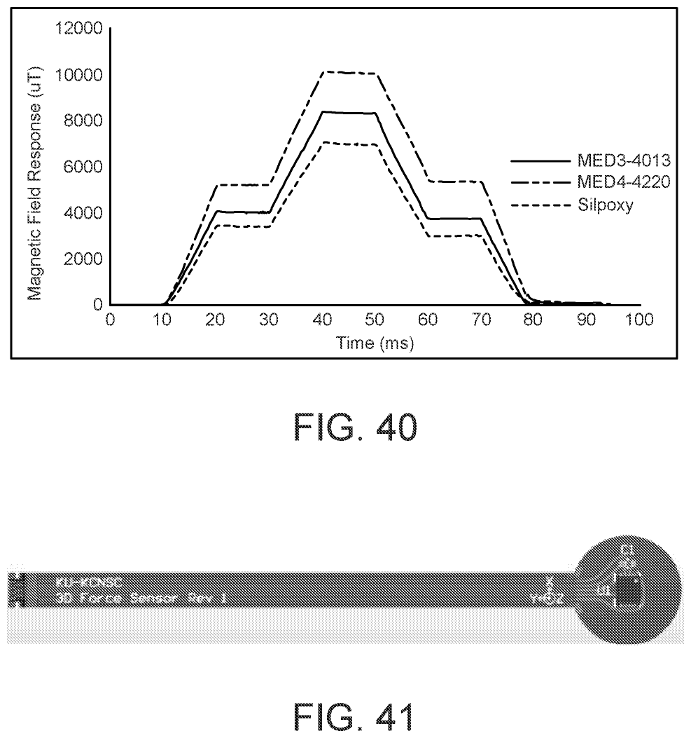

Sensors were constructed by bonding the silicone sensor component to magnetometer circuit boards using Silpoxy, NuSil MED3-4013, and NuSil MED4-4220. Each sensor unit was subjected to a stepwise compression cycle to demonstrate this sensor platform can function adequately using several different adhesives. The sensors were compressed in increments of 0.5 mm to a total of 1 mm, which was a 33% strain of the rubber component of the sensor (3 mm height), and the compression was removed in the same stepwise fashion (FIG. 40). These data demonstrate the sensor platform can operate correctly when the silicone sensor component is bonded to the magnetometer using several different adhesives, but also provides evidence that the properties of the adhesive impact the sensitivity of the sensor, as softer adhesives yielded stronger signals from compression. The peak magnitude of the magnetic signal from the 1 mm compressions were 10,097 μT, 8,395 μT, and 7,059 μT for MED4-4220 (durometer=17 A), MED3-4013 (20 A), and Silpoxy (40 A) respectively.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A force sensor comprising:
a magnetic actuator having a proximal end and a distal end, the magnetic actuator comprising an elastomeric resin; and a population of magnetic particles dispersed within the elastomeric resin;
a magnetometer operatively positioned in proximity to the distal end of the magnetic actuator; and
a spacer disposed between the magnetometer and the distal end of the magnetic actuator, thereby creating a standoff distance between the magnetometer and the distal end of the magnetic actuator;
wherein the magnetic actuator and the magnetometer are sized relative to one another such that a force applied to the magnetic actuator in a x-y plane relative to the magnetometer, along a z-axis relative to the magnetometer, or any combination thereof produces a magnetic field response that is increasing and proportionate or decreasing and proportionate to the applied force.

2. The sensor of claim 1, wherein the magnetic actuator and the magnetometer are sized relative to one another such that compression of the magnetic actuator under an applied force along the z-axis relative to the magnetometer produces a magnetic field response that is increasing and proportionate or decreasing and proportionate to the applied force.

3. The sensor of any one of claims 1-2, wherein the magnetometer produces a magnetic field response that is increasing and proportionate or decreasing and proportionate to the applied force.

4. The sensor of any one of claim 1-4, wherein the magnetic actuator has a largest cross-sectional dimension that is smaller than a largest cross-sectional dimension of the magnetometer when the magnetic actuator is not subjected to an applied force.

5. The sensor of claim 4, wherein the largest cross-sectional dimension of the magnetic actuator is from 5% to 80% of the largest cross-sectional dimension of the magnetometer when the magnetic actuator is not subjected to an applied force.

6. The sensor of any of claims 1-5, wherein the magnetic actuator has a largest cross-sectional dimension that is smaller than a largest cross-sectional dimension of the magnetometer when the magnetic actuator is compressed by 40% under an applied force.

7. The sensor of claim 6, wherein the largest cross-sectional dimension of the magnetic actuator is from 50% to 90% of the largest cross-sectional dimension of the magnetometer when the magnetic actuator is compressed by 40% under an applied force.

8. The sensor of any of claims 1-7, wherein the magnetic actuator has a largest cross-sectional area that is smaller than a largest cross-sectional area of the magnetometer when the magnetic actuator is not subjected to an applied force.

9. The sensor of claim 8, wherein the largest cross-sectional area of the magnetic actuator is from 50% to 90% of the largest cross-sectional are of the magnetometer when the magnetic actuator is not subjected to an applied force.

10. The sensor of any of claims 1-9, wherein the magnetic actuator has a largest cross-sectional area that is smaller than a largest cross-sectional area of the magnetometer when the magnetic actuator is compressed by 40% under an applied force.

11. The sensor of claim 10, wherein the largest cross-sectional area of the magnetic actuator is from 50% to 90% of the largest cross-sectional area of the magnetometer when the magnetic actuator is compressed by 40% under an applied force.

12. The sensor of any of claims 1-11, wherein the magnetic actuator and the magnetometer are sized relative to one another such that compression of the magnetic actuator under a window of applied forces ranging from an applied force effective to compress the magnetic actuator by 5% to an applied force effective to compress the magnetic actuator by 40% produces a magnetic field response that is increasing and proportionate or decreasing and proportionate to the applied force across the window of applied forces.

13. The sensor of any of claims 1-12, wherein a force applied to the magnetic actuator in the x-y plane relative to the magnetometer produces a magnetic field response that is increasing and proportionate or decreasing and proportionate to the applied force, wherein a force applied to the magnetic actuator along the z-axis relative to the magnetometer produces a magnetic field response that is increasing and proportionate or decreasing and proportionate to the applied force, or any combination thereof.

14. The sensor of any of claims 1-12, wherein the magnetic actuator and the magnetometer are sized relative to one another such that compression of the magnetic actuator under a window of applied forces ranging from an applied force effective to compress the magnetic actuator by 3% to an applied force effective to compress the magnetic actuator by 20% produces a magnetic field response that is increasing and proportionate or decreasing and proportionate to the applied force across the window of applied forces.

15. The sensor of claim 1, wherein the magnetic actuator and the magnetometer are sized relative to one another such that shear of the magnetic actuator under an applied force in a x-y plane relative to the magnetometer produces a magnetic field response that is increasing and proportionate or decreasing and proportionate to the applied force.

16. The sensor of claim 15, wherein the magnetometer produces a magnetic field response that is increasing and proportionate or decreasing and proportionate to the applied force.

17. The sensor of any one of claim 15-16, wherein the magnetic actuator has a largest cross-sectional dimension that is smaller than a largest cross-sectional dimension of the magnetometer when the magnetic actuator is not subjected to an applied force.

18. The sensor of claim 17, wherein the largest cross-sectional dimension of the magnetic actuator is from 5% to 80% of the largest cross-sectional dimension of the magnetometer when the magnetic actuator is not subjected to an applied force.

19. The sensor of any of claims 15-18, wherein the magnetic actuator has a largest cross-sectional dimension that is smaller than a largest cross-sectional dimension of the magnetometer when the magnetic actuator is sheared by 40% under an applied force.

20. The sensor of claim 19, wherein the largest cross-sectional dimension of the magnetic actuator is from 50% to 90% of the largest cross-sectional dimension of the magnetometer when the magnetic actuator is sheared by 40% under an applied force.

21. The sensor of any of claims 15-20, wherein the magnetic actuator has a largest cross-sectional area that is smaller than a largest cross-sectional area of the magnetometer when the magnetic actuator is not subjected to an applied force.

22. The sensor of claim 21, wherein the largest cross-sectional area of the magnetic actuator is from 50% to 90% of the largest cross-sectional are of the magnetometer when the magnetic actuator is not subjected to an applied force.

23. The sensor of any of claims 15-22, wherein the magnetic actuator has a largest cross-sectional area that is smaller than a largest cross-sectional area of the magnetometer when the magnetic actuator is sheared by 40% under an applied force.

24. The sensor of claim 23, wherein the largest cross-sectional area of the magnetic actuator is from 50% to 90% of the largest cross-sectional area of the magnetometer when the magnetic actuator is sheared by 40% under an applied force.

25. The sensor of any of claims 15-24, wherein the magnetic actuator and the magnetometer are sized relative to one another such that shear of the magnetic actuator under a window of applied forces ranging from an applied force effective to induce a shear strain of the magnetic actuator by 5% to an applied force effective to induce a shear strain of the magnetic actuator by 40% produces a magnetic field response that is increasing and proportionate or decreasing and proportionate to the applied force across the window of applied forces.

26. The sensor of any of claims 15-25, wherein the magnetic actuator and the magnetometer are sized relative to one another such that shear of the magnetic actuator under a window of applied forces ranging from an applied force effective to induce a shear strain of the magnetic actuator by 3% to an applied force effective to induce a shear strain of the magnetic actuator by 20% produces a magnetic field response that is increasing and proportionate or decreasing and proportionate to the applied force across the window of applied forces. 27 The sensor of any of claims 1-26, wherein the magnetic actuator has a largest cross-sectional dimension of from 1 mm to 25 mm.

28. The sensor of any of claims 1-26, wherein the magnetic actuator has a substantially circular horizontal cross-section.

29. The sensor of any of claims 1-28, wherein the magnetic actuator has a substantially cylindrical shape or a substantially conical shape.

30. The sensor of any of claims 1-29, wherein the standoff distance is from greater than 0 mm to 5 mm, such as from greater than 0 mm to 1.5 mm.

31. The sensor of any of claims 1-30, wherein the standoff distance is selected to provide a measurable signal such as magnetic field response greater than 100 μT with an applied force.

32. The sensor of any of claims 1-31, wherein the spacer is formed from an elastomeric resin, a rigid material, or any combination thereof.

33. The sensor of any of claims 1-32, wherein the spacer is formed from an elastomeric resin.

34. The sensor of any of claims 1-33, wherein when the spacer is formed from an elastomeric resin, the spacer comprises a portion of a housing that partially or completely encloses the magnetic actuator.

35. The sensor of any of claims 1-34, wherein the magnetic particles comprise magnetic microparticles.

36. The sensor of claim 35, wherein the magnetic microparticles have an average particle size of from 1 micron to 150 microns, such as from 1 micron to 50 microns.

37. The sensor of any of claims 1-36, wherein the magnetic particles comprise magnetic nanoparticles.

38. The sensor of claim 37, wherein the magnetic nanoparticles have an average particle size of from 50 nm to less than 1 micron, such as from 50 nm to 500 nm.

39. The sensor of any of claims 1-38, wherein the magnetic particles comprise anisotropic magnetic particles.

40. The sensor of any of claims 1-39, wherein the magnetic particles are present in the elastomeric resin in an amount of from 0.1% by weight to 90% by weight, based on the total weight of the elastomeric resin, such as from 50% by weight to 90% by weight, from 40% by weight to 80% by weight, from 30% to 70% by weight, from 20% to 60% by weight, from 15% to 50% by weight, from 0.1% to 50% by weight, from 0.1% to 40% by weight, from 0.1% to 30% by weight, from 0.1% to 20% by weight, from 0.1% by weight to 10% by weight, 0.1% by weight to 5% by weight, from 0.1% by weight to 2.5% by weight, or from 0.1% by weight to 1% by weight, based on the total weight of the elastomeric resin.

41. The sensor of any of claims 1-40, wherein the elastomeric resin further comprises a non-magnetic filler, such as silica particles.

42. The sensor of any of claims 1-41, wherein the elastomeric resin comprises a crosslinkable composition, such as a crosslinkable silicone composition.

43. The sensor of claim 42, wherein the elastomeric resin comprises (A) a first organosilicon compound having at least two ethylenically unsaturated moieties per molecule; and optionally (B) one or more additional organosilicon compounds.

44. The sensor of any of claims 1-43, wherein dipoles of the magnetic particles are aligned and/or oriented within the magnetic actuator.

45. The sensor of claim 44, wherein dipoles of the magnetic particles are aligned and/or oriented within the magnetic actuator when the magnetic actuator is compressed by from 10% to 60% under an applied force.

46. The sensor of any of claims 1-45, wherein the sensor further comprises a microcontroller, a processor, or a combination thereof operatively coupled to the magnetometer and configured to calculate a force applied to the magnetometer based on a measurement of a change in magnetic field strength.

47. A force sensor comprising:
a magnetic actuator having a proximal end and a distal end;
a magnetometer operatively positioned in proximity to the distal end of the magnetic actuator; and
an elastomeric spacer disposed between the magnetometer and the distal end of the magnetic actuator, thereby creating a standoff distance between the magnetometer and the distal end of the magnetic actuator;
wherein the magnetic actuator and the magnetometer are sized relative to one another such that a force applied to the magnetic actuator in a x-y plane relative to the magnetometer, along a z-axis relative to the magnetometer, or any combination thereof produces a magnetic field response that is increasing and proportionate or decreasing and proportionate to the applied force.

48. The sensor of claim 47, wherein the elastomeric spacer is formed from an elastomeric resin.

49. The sensor of any of claims 47-48, wherein the elastomeric spacer comprises a portion of a housing that partially or completely encloses the magnetic actuator.

50. The sensor of any of claims 47-49, further comprising a rigid spacer disposed between the magnetometer and the distal end of the elastomeric spacer, thereby creating a standoff distance between the magnetometer and the distal end of the elastomeric housing, wherein the rigid spacer is formed from a rigid material such as hard plastic, wood, glass, non-magnetic metal, or a material with a Shore A Hardness of greater than 70 and/or a Shore D Hardness of greater than 10.

51. A force sensor comprising:
a magnetic actuator having a proximal end and a distal end;
a magnetometer operatively positioned in proximity to the distal end of the magnetic actuator; and
an elastomeric housing enclosing at least a portion of the magnetic actuator and extending beyond the distal end of the magnetic actuator, thereby creating a standoff distance between the magnetometer and the distal end of the magnetic actuator;
wherein the magnetic actuator and the magnetometer are sized relative to one another such that a force applied to the magnetic actuator in a x-y plane relative to the magnetometer, along a z-axis relative to the magnetometer, or any combination thereof produces a magnetic field response that is increasing and proportionate or decreasing and proportionate to the applied force.

52. The sensor of any of claims 47-51, wherein the magnetic actuator and the magnetometer are sized relative to one another such that compression of the magnetic actuator under an applied force along the z-axis relative to the magnetometer produces a magnetic field response that is increasing and proportionate or decreasing and proportionate to the applied force.

53. The sensor of any one of claims 47-52, wherein the magnetometer produces a magnetic field response that is increasing and proportionate or decreasing and proportionate to the applied force.

54. The sensor of any one of claim 47-53, wherein the magnetic actuator has a largest cross-sectional dimension that is smaller than a largest cross-sectional dimension of the magnetometer when the magnetic actuator is not subjected to an applied force.

55. The sensor of claim 54, wherein the largest cross-sectional dimension of the magnetic actuator is from 5% to 80% of the largest cross-sectional dimension of the magnetometer when the magnetic actuator is not subjected to an applied force.

56. The sensor of any of claims 47-55, wherein the magnetic actuator has a largest cross-sectional dimension that is smaller than a largest cross-sectional dimension of the magnetometer when the magnetic actuator is compressed by 40% under an applied force.

57. The sensor of claim 56, wherein the largest cross-sectional dimension of the magnetic actuator is from 50% to 90% of the largest cross-sectional dimension of the magnetometer when the magnetic actuator is compressed by 40% under an applied force.

58. The sensor of any of claims 47-57, wherein the magnetic actuator has a largest cross-sectional area that is smaller than a largest cross-sectional area of the magnetometer when the magnetic actuator is not subjected to an applied force.

59. The sensor of claim 58, wherein the largest cross-sectional area of the magnetic actuator is from 50% to 90% of the largest cross-sectional are of the magnetometer when the magnetic actuator is not subjected to an applied force.

60. The sensor of any of claims 47-59, wherein the magnetic actuator has a largest cross-sectional area that is smaller than a largest cross-sectional area of the magnetometer when the magnetic actuator is compressed by 40% under an applied force.

61. The sensor of claim 60, wherein the largest cross-sectional area of the magnetic actuator is from 50% to 90% of the largest cross-sectional area of the magnetometer when the magnetic actuator is compressed by 40% under an applied force.

62. The sensor of any of claims 47-61, wherein the magnetic actuator and the magnetometer are sized relative to one another such that compression of the magnetic actuator under a window of applied forces ranging from an applied force effective to compress the magnetic actuator by 5% to an applied force effective to compress the magnetic actuator by 40% produces a magnetic field response that is increasing and proportionate or decreasing and proportionate to the applied force across the window of applied forces.

63. The sensor of any of claims 47-62, wherein a force applied to the magnetic actuator in the x-y plane relative to the magnetometer produces a magnetic field response that is increasing and proportionate or decreasing and proportionate to the applied force, wherein a force applied to the magnetic actuator along the z-axis relative to the magnetometer produces a magnetic field response that is increasing and proportionate or decreasing and proportionate to the applied force, or any combination thereof.

64. The sensor of any of claims 47-63, wherein the magnetic actuator and the magnetometer are sized relative to one another such that compression of the magnetic actuator under a window of applied forces ranging from an applied force effective to compress the magnetic actuator by 3% to an applied force effective to compress the magnetic actuator by 20% produces a magnetic field response that is increasing and proportionate or decreasing and proportionate to the applied force across the window of applied forces.

65. The sensor of any of claims 47-51, wherein the magnetic actuator and the magnetometer are sized relative to one another such that shear of the magnetic actuator under an applied force in a x-y plane relative to the magnetometer produces a magnetic field response that is increasing and proportionate or decreasing and proportionate to the applied force.

66. The sensor of claim 65, wherein the magnetometer produces a magnetic field response that is increasing and proportionate or decreasing and proportionate to the applied force.

67. The sensor of any one of claim 65-66, wherein the magnetic actuator has a largest cross-sectional dimension that is smaller than a largest cross-sectional dimension of the magnetometer when the magnetic actuator is not subjected to an applied force.

68. The sensor of claim 67, wherein the largest cross-sectional dimension of the magnetic actuator is from 5% to 80% of the largest cross-sectional dimension of the magnetometer when the magnetic actuator is not subjected to an applied force.

69. The sensor of any of claims 65-68, wherein the magnetic actuator has a largest cross-sectional dimension that is smaller than a largest cross-sectional dimension of the magnetometer when the magnetic actuator is sheared by 40% under an applied force.

70. The sensor of claim 69, wherein the largest cross-sectional dimension of the magnetic actuator is from 50% to 90% of the largest cross-sectional dimension of the magnetometer when the magnetic actuator is sheared by 40% under an applied force.

71. The sensor of any of claims 65-70, wherein the magnetic actuator has a largest cross-sectional area that is smaller than a largest cross-sectional area of the magnetometer when the magnetic actuator is not subjected to an applied force.

72. The sensor of claim 71, wherein the largest cross-sectional area of the magnetic actuator is from 50% to 90% of the largest cross-sectional are of the magnetometer when the magnetic actuator is not subjected to an applied force.

73. The sensor of any of claims 65-72, wherein the magnetic actuator has a largest cross-sectional area that is smaller than a largest cross-sectional area of the magnetometer when the magnetic actuator is sheared by 40% under an applied force.

74. The sensor of claim 73, wherein the largest cross-sectional area of the magnetic actuator is from 50% to 90% of the largest cross-sectional area of the magnetometer when the magnetic actuator is sheared by 40% under an applied force.

75. The sensor of any of claims 65-74, wherein the magnetic actuator and the magnetometer are sized relative to one another such that shear of the magnetic actuator under a window of applied forces ranging from an applied force effective to induce a shear strain of the magnetic actuator by 5% to an applied force effective to induce a shear strain of the magnetic actuator by 40% produces a magnetic field response that is increasing and proportionate or decreasing and proportionate to the applied force across the window of applied forces.

76. The sensor of any of claims 65-75, wherein the magnetic actuator and the magnetometer are sized relative to one another such that shear of the magnetic actuator under a window of applied forces ranging from an applied force effective to induce a shear strain of the magnetic actuator by 3% to an applied force effective to induce a shear strain of the magnetic actuator by 20% produces a magnetic field response that is increasing and proportionate or decreasing and proportionate to the applied force across the window of applied forces.

77. The sensor of any of claims 47-76, wherein the magnetic actuator has a largest cross-sectional dimension of from 1 mm to 25 mm.

78. The sensor of any of claims 47-76, wherein the magnetic actuator has a substantially circular horizontal cross-section.

79. The sensor of any of claims 47-78, wherein the magnetic actuator has a substantially cylindrical shape or a substantially conical shape.

80. The sensor of any of claims 47-79, wherein the standoff distance is from greater than 0 mm to 5 mm, such as from greater than 0 mm to 1.5 mm.

81. The sensor of any of claims 1-30, wherein the standoff distance is selected to provide a measurable signal such as magnetic field response greater than 100 μT with an applied force.

82. The sensor of any of claims 47-81, further comprising a rigid spacer disposed between the magnetometer and the distal end of the elastomeric housing, thereby creating a standoff distance between the magnetometer and the distal end of the elastomeric housing, wherein the rigid spacer is formed from a rigid material such as hard plastic, wood, glass, non-magnetic metal, or a material with a Shore A Hardness of greater than 70 and/or a Shore D Hardness of greater than 10.

83. The sensor of any of claims 47-82, wherein the elastomeric housing is formed from an elastomeric resin.

84. The sensor of any of claims 47-83, wherein the magnetic actuator comprises an elastomeric resin; and a population of magnetic particles dispersed within the elastomeric resin.

85. The sensor of claim 84, wherein the magnetic particles comprise magnetic microparticles.

86. The sensor of claim 85, wherein the magnetic microparticles have an average particle size of from 1 micron to 150 microns, such as from 1 micron to 50 microns.

87. The sensor of claim 84, wherein the magnetic particles comprise magnetic nanoparticles.

88. The sensor of claim 87, wherein the magnetic nanoparticles have an average particle size of from 50 nm to less than 1 micron, such as from 50 nm to 500 nm.

89. The sensor of any of claims 84, wherein the magnetic particles comprise anisotropic magnetic particles.

90. The sensor of any of claims 47-89, wherein the magnetic particles are present in the elastomeric resin in an amount of from 0.1% by weight to 90% by weight, based on the total weight of the elastomeric resin, such as from 50% by weight to 90% by weight, from 40% by weight to 80% by weight, from 30% to 70% by weight, from 20% to 60% by weight, from 15% to 50% by weight, from 0.1% to 50% by weight, from 0.1% to 40% by weight, from 0.1% to 30% by weight, from 0.1% to 20% by weight, from 0.1% by weight to 10% by weight, 0.1% by weight to 5% by weight, from 0.1% by weight to 2.5% by weight, or from 0.1% by weight to 1% by weight, based on the total weight of the elastomeric resin.

91. The sensor of any of claims 47-90, wherein the elastomeric resin further comprises a non-magnetic filler, such as silica particles.

92. The sensor of any of claims 47-91, wherein the elastomeric resin comprises a crosslinkable composition, such as a crosslinkable silicone composition.

93. The sensor of claim 92, wherein the elastomeric resin comprises (A) a first organosilicon compound having at least two ethylenically unsaturated moieties per molecule; and optionally (B) one or more additional organosilicon compounds.

94. The sensor of any of claims 84-93, wherein dipoles of the magnetic particles are aligned and/or oriented within the magnetic actuator.

95. The sensor of claim 94, wherein dipoles of the magnetic particles are aligned and/or oriented within the magnetic actuator when the magnetic actuator is compressed by from 10% to 60% under an applied force.

96. The sensor of any of claims 47-95, wherein the sensor further comprises a microcontroller, a processor, or a combination thereof operatively coupled to the magnetometer and configured to calculate a force applied to the magnetometer based on a measurement of a change in magnetic field strength.

97. A force sensor comprising:
two or more magnetic actuators, each magnetic actuator having a proximal end and a distal end;
a magnetometer operatively positioned in proximity to the distal end of the one or more magnetic actuators; and
a spacer disposed between the magnetometer and the distal end of the one or more magnetic actuators, thereby creating a standoff distance between the magnetometer and the distal end of the one or more magnetic actuators;
wherein the two or more magnetic actuators and the magnetometer are sized relative to one another such that a force applied to the two or more magnetic actuators in a x-y plane relative to the magnetometer, along a z-axis relative to the magnetometer, or any combination thereof produces a magnetic field response that is increasing and proportionate or decreasing and proportionate to the applied force.

98. The sensor of claim 97, wherein each of the magnetic actuators are adjacent to each other.

99. The sensor of any of claims 97-98, wherein the magnetometer is operatively positioned in proximity to the distal end of the magnetic actuators.

100. The sensor of any of claims 97-99, further comprising a rigid spacer disposed between the magnetometer and the distal end of the two or more magnetic actuators, thereby creating a distance between the magnetometer and the distal end of the two or more magnetic actuators, wherein the rigid spacer is formed from a rigid material such as hard plastic, wood, glass, non-magnetic metal, or a material with a Shore A Hardness of greater than 70 and/or a Shore D Hardness of greater than 10.

101. A force sensor comprising:
a magnetic actuator, having a proximal end and a distal end;
two or more magnetometers operatively positioned in proximity to the distal end of the magnetic actuator; and
a spacer disposed between the two or more magnetometers and the distal end of the magnetic actuator, thereby creating a standoff distance between the one or more magnetometers and the distal end of the magnetic actuator;
wherein the magnetic actuator and the two or more magnetometers are sized relative to one another such that a force applied to the magnetic actuator in a x-y plane relative to the two or more magnetometers, along a z-axis relative to the two or more magnetometers, or any combination thereof produces a magnetic field response that is increasing and proportionate or decreasing and proportionate to the applied force.

102. The sensor of claim 101, wherein each of the magnetometers are adjacent to each other.

103. The sensor of claim 97-102, wherein the magnetic actuator and the magnetometer are sized relative to one another such that compression of the magnetic actuator under an applied force along the z-axis relative to the magnetometer produces a magnetic field response that is increasing and proportionate or decreasing and proportionate to the applied force.

104. The sensor of any one of claims 97-103, wherein the magnetometer produces a magnetic field response that is increasing and proportionate or decreasing and proportionate to the applied force.

105. The sensor of any one of claim 97-103, wherein the magnetic actuator has a largest cross-sectional dimension that is smaller than a largest cross-sectional dimension of the magnetometer when the magnetic actuator is not subjected to an applied force.

106. The sensor of claim 105, wherein the largest cross-sectional dimension of the magnetic actuator is from 5% to 80% of the largest cross-sectional dimension of the magnetometer when the magnetic actuator is not subjected to an applied force.

107. The sensor of any of claims 97-105, wherein the magnetic actuator has a largest cross-sectional dimension that is smaller than a largest cross-sectional dimension of the magnetometer when the magnetic actuator is compressed by 40% under an applied force.

108. The sensor of claim 107, wherein the largest cross-sectional dimension of the magnetic actuator is from 50% to 90% of the largest cross-sectional dimension of the magnetometer when the magnetic actuator is compressed by 40% under an applied force.

109. The sensor of any of claims 97-108, wherein the magnetic actuator has a largest cross-sectional area that is smaller than a largest cross-sectional area of the magnetometer when the magnetic actuator is not subjected to an applied force.

110. The sensor of claim 109, wherein the largest cross-sectional area of the magnetic actuator is from 50% to 90% of the largest cross-sectional are of the magnetometer when the magnetic actuator is not subjected to an applied force.

111. The sensor of any of claims 97-109, wherein the magnetic actuator has a largest cross-sectional area that is smaller than a largest cross-sectional area of the magnetometer when the magnetic actuator is compressed by 40% under an applied force.

112. The sensor of claim 111, wherein the largest cross-sectional area of the magnetic actuator is from 50% to 90% of the largest cross-sectional area of the magnetometer when the magnetic actuator is compressed by 40% under an applied force.

113. The sensor of any of claims 97-112, wherein the magnetic actuator and the magnetometer are sized relative to one another such that compression of the magnetic actuator under a window of applied forces ranging from an applied force effective to compress the magnetic actuator by 5% to an applied force effective to compress the magnetic actuator by 40% produces a magnetic field response that is increasing and proportionate or decreasing and proportionate to the applied force across the window of applied forces.

114. The sensor of any of claims 97-113, wherein a force applied to the magnetic actuator in the x-y plane relative to the two or more magnetometers produces a magnetic field response that is increasing and proportionate or decreasing and proportionate to the applied force, wherein a force applied to the magnetic actuator along the z-axis relative to the two or more magnetometers produces a magnetic field response that is increasing and proportionate or decreasing and proportionate to the applied force, or any combination thereof.

115. The sensor of any of claims 97-114, wherein the magnetic actuator and the magnetometer are sized relative to one another such that compression of the magnetic actuator under a window of applied forces ranging from an applied force effective to compress the magnetic actuator by 3% to an applied force effective to compress the magnetic actuator by 20% produces a magnetic field response that is increasing and proportionate or decreasing and proportionate to the applied force across the window of applied forces.

116. The sensor of claim 97, wherein the magnetic actuator and the magnetometer are sized relative to one another such that shear of the magnetic actuator under an applied force in a x-y plane relative to the magnetometer produces a magnetic field response that is increasing and proportionate or decreasing and proportionate to the applied force.

117. The sensor of claim 116, wherein the magnetometer produces a magnetic field response that is increasing and proportionate or decreasing and proportionate to the applied force.

118. The sensor of any one of claim 116-117, wherein the magnetic actuator has a largest cross-sectional dimension that is smaller than a largest cross-sectional dimension of the magnetometer when the magnetic actuator is not subjected to an applied force.

119. The sensor of claim 118, wherein the largest cross-sectional dimension of the magnetic actuator is from 5% to 80% of the largest cross-sectional dimension of the magnetometer when the magnetic actuator is not subjected to an applied force.

120. The sensor of any of claims 116-119, wherein the magnetic actuator has a largest cross-sectional dimension that is smaller than a largest cross-sectional dimension of the magnetometer when the magnetic actuator is sheared by 40% under an applied force.

121. The sensor of claim 120, wherein the largest cross-sectional dimension of the magnetic actuator is from 50% to 90% of the largest cross-sectional dimension of the magnetometer when the magnetic actuator is sheared by 40% under an applied force.

122. The sensor of any of claims 116-121, wherein the magnetic actuator has a largest cross-sectional area that is smaller than a largest cross-sectional area of the magnetometer when the magnetic actuator is not subjected to an applied force.

123. The sensor of claim 122, wherein the largest cross-sectional area of the magnetic actuator is from 50% to 90% of the largest cross-sectional are of the magnetometer when the magnetic actuator is not subjected to an applied force.

124. The sensor of any of claims 116-123, wherein the magnetic actuator has a largest cross-sectional area that is smaller than a largest cross-sectional area of the magnetometer when the magnetic actuator is sheared by 40% under an applied force.

125. The sensor of claim 124, wherein the largest cross-sectional area of the magnetic actuator is from 50% to 90% of the largest cross-sectional area of the magnetometer when the magnetic actuator is sheared by 40% under an applied force.

126. The sensor of any of claims 116-125, wherein the magnetic actuator and the magnetometer are sized relative to one another such that shear of the magnetic actuator under a window of applied forces ranging from an applied force effective to induce a shear strain of the magnetic actuator by 5% to an applied force effective to induce a shear strain of the magnetic actuator by 40% produces a magnetic field response that is increasing and proportionate or decreasing and proportionate to the applied force across the window of applied forces.

127. The sensor of any of claims 116-126, wherein the magnetic actuator and the magnetometer are sized relative to one another such that shear of the magnetic actuator under a window of applied forces ranging from an applied force effective to induce a shear strain of the magnetic actuator by 3% to an applied force effective to induce a shear strain of the magnetic actuator by 20% produces a magnetic field response that is increasing and proportionate or decreasing and proportionate to the applied force across the window of applied forces.

128. The sensor of any of claims 97-127, wherein the spacer is formed from an elastomeric resin, a rigid material, or any combination thereof.

129. The sensor of any of claims 97-128, wherein the spacer is formed from an elastomeric resin.

130. The sensor of any of claims 97-129, wherein when the spacer is formed from an elastomeric resin, the spacer comprises a portion of a housing that partially or completely encloses the magnetic actuator.

131. The sensor of any of claims 97-130, further comprising a rigid spacer disposed between the two or more magnetometers and the distal end of the magnetic actuator, thereby creating a distance between the two or more magnetometers and the distal end of the magnetic actuator, wherein the rigid spacer is formed from a rigid material such as hard plastic, wood, glass, non-magnetic metal, or a material with a Shore A Hardness of greater than 70 and/or a Shore D Hardness of greater than 10.

132. A force sensor comprising:
a magnetic actuator, having a proximal end and a distal end;
a magnetometer operatively positioned in proximity to the distal end of the magnetic actuator; and
an elastomeric housing enclosing at least a portion of the magnetic actuator and extending beyond the distal end of the magnetic actuator, thereby creating a standoff distance between the magnetometer and the distal end of the magnetic actuator;
wherein no elastomeric housing is disposed between the magnetometer and the distal end of the magnetic actuator;
wherein the magnetic actuator and the magnetometer are sized relative to one another such that a force applied to the magnetic actuator in a x-y plane relative to the magnetometer, along a z-axis relative to the magnetometer, or any combination thereof produces a magnetic field response that is increasing and proportionate or decreasing and proportionate to the applied force.

133. The sensor of claim 132, wherein the magnetic actuator and the magnetometer are sized relative to one another such that compression of the magnetic actuator under an applied force along the z-axis relative to the magnetometer produces a magnetic field response that is increasing and proportionate or decreasing and proportionate to the applied force.

134. The sensor of any one of claims 132-133, wherein the magnetometer produces a magnetic field response that is increasing and proportionate or decreasing and proportionate to the applied force.

135. The sensor of any one of claim 132-134, wherein the magnetic actuator has a largest cross-sectional dimension that is smaller than a largest cross-sectional dimension of the magnetometer when the magnetic actuator is not subjected to an applied force.

136. The sensor of claim 135, wherein the largest cross-sectional dimension of the magnetic actuator is from 5% to 80% of the largest cross-sectional dimension of the magnetometer when the magnetic actuator is not subjected to an applied force.

137. The sensor of any of claims 132-136, wherein the magnetic actuator has a largest cross-sectional dimension that is smaller than a largest cross-sectional dimension of the magnetometer when the magnetic actuator is compressed by 40% under an applied force.

138. The sensor of claim 137, wherein the largest cross-sectional dimension of the magnetic actuator is from 50% to 90% of the largest cross-sectional dimension of the magnetometer when the magnetic actuator is compressed by 40% under an applied force.

139. The sensor of any of claims 132-138, wherein the magnetic actuator has a largest cross-sectional area that is smaller than a largest cross-sectional area of the magnetometer when the magnetic actuator is not subjected to an applied force.

140. The sensor of claim 139, wherein the largest cross-sectional area of the magnetic actuator is from 50% to 90% of the largest cross-sectional are of the magnetometer when the magnetic actuator is not subjected to an applied force.

141. The sensor of any of claims 132-140, wherein the magnetic actuator has a largest cross-sectional area that is smaller than a largest cross-sectional area of the magnetometer when the magnetic actuator is compressed by 40% under an applied force.

142. The sensor of claim 141, wherein the largest cross-sectional area of the magnetic actuator is from 50% to 90% of the largest cross-sectional area of the magnetometer when the magnetic actuator is compressed by 40% under an applied force.

143. The sensor of any of claims 132-142, wherein the magnetic actuator and the magnetometer are sized relative to one another such that compression of the magnetic actuator under a window of applied forces ranging from an applied force effective to compress the magnetic actuator by 5% to an applied force effective to compress the magnetic actuator by 40% produces a magnetic field response that is increasing and proportionate or decreasing and proportionate to the applied force across the window of applied forces.

144. The sensor of any of claims 132-143, wherein a force applied to the magnetic actuator in the x-y plane relative to the magnetometer produces a magnetic field response that is increasing and proportionate or decreasing and proportionate to the applied force, wherein a force applied to the magnetic actuator along the z-axis relative to the magnetometer produces a magnetic field response that is increasing and proportionate or decreasing and proportionate to the applied force, or any combination thereof.

145. The sensor of any of claims 132-144, wherein the magnetic actuator and the magnetometer are sized relative to one another such that compression of the magnetic actuator under a window of applied forces ranging from an applied force effective to compress the magnetic actuator by 3% to an applied force effective to compress the magnetic actuator by 20% produces a magnetic field response that is increasing and proportionate or decreasing and proportionate to the applied force across the window of applied forces.

146. The sensor of any of claims 132-145, wherein the magnetic actuator and the magnetometer are sized relative to one another such that shear of the magnetic actuator under an applied force in a x-y plane relative to the magnetometer produces a magnetic field response that is increasing and proportionate or decreasing and proportionate to the applied force.

147. The sensor of claim 146, wherein the magnetometer produces a magnetic field response that is increasing and proportionate or decreasing and proportionate to the applied force.

148. The sensor of any one of claim 132-147, wherein the magnetic actuator has a largest cross-sectional dimension that is smaller than a largest cross-sectional dimension of the magnetometer when the magnetic actuator is not subjected to an applied force.

149. The sensor of claim 148, wherein the largest cross-sectional dimension of the magnetic actuator is from 5% to 80% of the largest cross-sectional dimension of the magnetometer when the magnetic actuator is not subjected to an applied force.

150. The sensor of any of claims 132-148, wherein the magnetic actuator has a largest cross-sectional dimension that is smaller than a largest cross-sectional dimension of the magnetometer when the magnetic actuator is sheared by 40% under an applied force.

151. The sensor of claim 150, wherein the largest cross-sectional dimension of the magnetic actuator is from 50% to 90% of the largest cross-sectional dimension of the magnetometer when the magnetic actuator is sheared by 40% under an applied force.

152. The sensor of any of claims 132-151, wherein the magnetic actuator has a largest cross-sectional area that is smaller than a largest cross-sectional area of the magnetometer when the magnetic actuator is not subjected to an applied force.

153. The sensor of claim 152, wherein the largest cross-sectional area of the magnetic actuator is from 50% to 90% of the largest cross-sectional are of the magnetometer when the magnetic actuator is not subjected to an applied force.

154. The sensor of any of claims 132-153, wherein the magnetic actuator has a largest cross-sectional area that is smaller than a largest cross-sectional area of the magnetometer when the magnetic actuator is sheared by 40% under an applied force.

155. The sensor of claim 154, wherein the largest cross-sectional area of the magnetic actuator is from 50% to 90% of the largest cross-sectional area of the magnetometer when the magnetic actuator is sheared by 40% under an applied force.

156. The sensor of any of claims 132-155, wherein the magnetic actuator and the magnetometer are sized relative to one another such that shear of the magnetic actuator under a window of applied forces ranging from an applied force effective to induce a shear strain of the magnetic actuator by 5% to an applied force effective to induce a shear strain of the magnetic actuator by 40% produces a magnetic field response that is increasing and proportionate or decreasing and proportionate to the applied force across the window of applied forces.

157. The sensor of any of claims 132-156, wherein the magnetic actuator and the magnetometer are sized relative to one another such that shear of the magnetic actuator under a window of applied forces ranging from an applied force effective to induce a shear strain of the magnetic actuator by 3% to an applied force effective to induce a shear strain of the magnetic actuator by 20% produces a magnetic field response that is increasing and proportionate or decreasing and proportionate to the applied force across the window of applied forces.

158. The sensor of any of claims 97-157, wherein the magnetic actuator has a largest cross-sectional dimension of from 1 mm to 25 mm.

159. The sensor of any of claims 97-158, wherein the magnetic actuator has a substantially circular horizontal cross-section.

160. The sensor of any of claims 97-159, wherein the magnetic actuator has a substantially cylindrical shape or a substantially conical shape.

161. The sensor of any of claims 97-160, wherein the standoff distance is from greater than 0 mm to 5 mm, such as from greater than 0 mm to 1.5 mm.

162. The sensor of any of claims 97-161, wherein the standoff distance is selected to provide a measurable signal such as magnetic field response greater than 100 μT with an applied force.

163. The sensor of any of claims 132-162, further comprising a rigid spacer disposed between the magnetometer and the distal end of the elastomeric housing, thereby creating a standoff distance between the magnetometer and the distal end of the elastomeric housing, wherein the rigid spacer is formed from a rigid material such as hard plastic, wood, glass, non-magnetic metal, or a material with a Shore A Hardness of greater than 70 and/or a Shore D Hardness of greater than 10.

164. The sensor of any of claims 132-163, wherein the elastomeric housing is formed from an elastomeric resin.

165. The sensor of any of claims 97-164, wherein the magnetic actuator comprises an elastomeric resin; and a population of magnetic particles dispersed within the elastomeric resin.

166. The sensor of claim 165, wherein the magnetic microparticles have an average particle size of from 1 micron to 150 microns, such as from 1 micron to 50 microns.

167. The sensor of any of claims 165-166, wherein the magnetic particles comprise magnetic nanoparticles.

168. The sensor of claim 167, wherein the magnetic nanoparticles have an average particle size of from 50 nm to less than 1 micron, such as from 50 nm to 500 nm.

169. The sensor of any of claims 165-168 wherein the magnetic particles comprise anisotropic magnetic particles.

170. The sensor of any of claims 165-169, wherein the magnetic particles are present in the elastomeric resin in an amount of from 0.1% by weight to 90% by weight, based on the total weight of the elastomeric resin, such as from 50% by weight to 90% by weight, from 40% by weight to 80% by weight, from 30% to 70% by weight, from 20% to 60% by weight, from 15% to 50% by weight, from 0.1% to 50% by weight, from 0.1% to 40% by weight, from 0.1% to 30% by weight, from 0.1% to 20% by weight, from 0.1% by weight to 10% by weight, 0.1% by weight to 5% by weight, from 0.1% by weight to 2.5% by weight, or from 0.1% by weight to 1% by weight, based on the total weight of the elastomeric resin.

171. The sensor of any of claims 97-170, wherein the elastomeric resin further comprises a non-magnetic filler, such as silica particles.

172. The sensor of any of claims 97-171, wherein the elastomeric resin comprises a crosslinkable composition, such as a crosslinkable silicone composition.

173. The sensor of claim 172, wherein the elastomeric resin comprises (A) a first organosilicon compound having at least two ethylenically unsaturated moieties per molecule; and optionally (B) one or more additional organosilicon compounds.

174. The sensor of any of claims 165-173, wherein dipoles of the magnetic particles are aligned and/or oriented within the magnetic actuator.

175. The sensor of claim 174, wherein dipoles of the magnetic particles are aligned and/or oriented within the magnetic actuator when the magnetic actuator is compressed by from 10% to 60% under an applied force.

176. The sensor of any of claims 97-175, wherein the sensor further comprises a microcontroller, a processor, or a combination thereof operatively coupled to the magnetometer and configured to calculate a force applied to the magnetometer based on a measurement of a change in magnetic field strength.
